(19) United States
(12) Patent Application Publication
(10) Pub. No.: US 2026/0191728 A1
(43) Pub. Date: Jul. 9, 2026

(54) SYSTEMS AND METHODS FOR MUSCLE-TENDON CONTROL OF WEARABLE-ROBOTIC DEVICES

(71) Applicant: Massachusetts Institute of Technology, Cambridge, MA (US)

(72) Inventors: Hugh M. Herr, Concord, NH (US); Jesus Guillermo Herrera-Arcos, Somerville, MA (US); Michael Thomas Nawrot, Cambridge, MA (US); Junqing Qiao, Cambridge, MA (US); Christopher Shallal, Cambridge, MA (US); Hyungeun Song, Cambridge, MA (US)

(21) Appl. No.: 19/131,898

(22) PCT Filed: Nov. 21, 2023
(86) PCT No.: PCT/US2023/080814

§ 371 Date: May 21, 2025

Related U.S. Application Data
(60) Provisional application No. 63/427,355, filed on Nov. 22, 2022.

(51) Int. Cl. A61H 3/00; A61B 8/00; A61B 8/08; A61B 90/00; A61H 1/02

(52) U.S. Cl. CPC A61H 3/00; A61B 8/08; A61B 8/485; A61B 8/5223; A61B 2090/3925; A61H 1/0266; A61H 2201/165; A61H 2201/5007; A61H 2230/605

(57) ABSTRACT

Devices, systems, and methods herein are generally directed to a robotic control system that comprises: a wearable robot comprising at least one actuated joint; at least one muscle-tendon interface configured to measure at least one physiological signal; a torque set point processor configured to receive the at least one physiological signal from the at least one muscle tendon interface and estimate a muscle-tendon state corresponding to a human motor intention and to compute at least one augmentation joint torque based on the muscle-tendon state; and a torque controller configured to apply the at least one augmentation joint torque to the at least one actuated joint of the wearable robot.

### RELATED APPLICATION

This application claims the benefit of U.S. Provisional Application No. 63/427,355, filed on Nov. 22, 2022. The entire teachings of the above application are incorporated herein by reference.

### BACKGROUND

Robotic exoskeletons offer the ability to improve motor impairments in individuals with neurological disorders due to stroke, multiple sclerosis, Parkinson's, or individuals with decreased functionality in muscles, tendons, ligaments, or their spinal cord due to injury or age-related degeneration. Beyond rehabilitation, exoskeletons can augment users beyond innate physiological capabilities by providing assistance at the joint level.

Exoskeleton control is challenging. To maintain health and vitality, the kinetic augmentation must cooperate and coordinate with the wearer's biological muscle torques and mechanical powers. Moreover, for real-world use beyond the laboratory, the controller needs to account for a variety of movements and gaits due to changes in the user's physiology (e.g., age, sex, muscle strength) and environment (e.g., different gait speeds, terrain types, or objects to manipulate) [1]. Current approaches rely on on-board robotic sensing or some type of brain-computer interface [2-8]. Methods that use on-board sensors often fail to determine the user's intention as they can only recognize a set of joint movements. Moreover, they require additional sensing of the environment (such as cameras or distance sensors) or must also employ machine learning combined with slow optimization algorithms. These methods do not operate in real time and large amounts of data are required to fully train or optimize exoskeletal torque profiles [10-13]. Current methods to extract the user's intent through brain-computer interfaces, such as electroencephalography (EEG) or electromyography (EMG), aren't reliable as signals are contaminated with other signals (e.g., EEG contaminated with EMG) and/or are slow (e.g., EEG), require multiple or bulky sensors (EEG, ultrasound probes), are limited to discontinuous trajectories, and require frequent re-calibration and training (EEG). No existing minimally-invasive neuromuscular interface delivers the intuitive control, adaptation, and natural reflexive response that are needed

### SUMMARY

Aspects of inventive concepts herein involve muscle-tendon interfaces, biophysical analyses, and controllers that address deficiencies previously mentioned. In some embodiments, real-time measurement of a muscle-tendon force, or its corresponding biological human joint torque contribution about a joint, serves as a real-time controller input for a closed-loop torque control system for a wearable robot acting in parallel to the biological joint spanned by the muscle-tendon unit. Such a direct, real-time neuromuscular control between biological muscle-tendon force, or its resulting biological joint torque, and wearable robotic torque is designed to deliver an intuitive control, adaptation and natural reflexive response without the need for complex and slow optimization routines.

In some example embodiments, aspects of inventive concepts herein involve a wearable robotic control strategy that uses one or more muscle-tendon interfaces to estimate one or more muscle-tendon forces, or one or more human biological torques. The at least one biological muscle-tendon force, or the at least one resulting biological joint torque, is used to compute an at least one augmentation torque. A torque controller then servos to the at least one augmentation torque to directly control the at least one output torque of a wearable robot (see FIG. 1). In various embodiments, a muscle-tendon interface presented herein measures in real time one or more muscle-tendon signals. Examples of muscle-tendon signals include but are not limited to muscle tissue strain, tendon strain, muscle velocity, tendon velocity, muscle speed, tendon speed, muscle activation, muscle stiffness, or a muscle-tendon moment arm (or moment arms) about a rotational joint axis (or axes). One or more of these parameters are then used to estimate muscle-tendon force, or its resulting biological human torque contribution. Augmentation torque about the joint is then estimated from a muscle-tendon force spanning that joint, or its resulting biological joint torque about that joint, and then used as a control target for an exoskeletal controller to modulate the corresponding device joint torque in parallel with the biological joint. In some control embodiments, the augmentation torque is functionally related to the muscle-tendon force, or its corresponding biological joint torque, linearly or nonlinearly.

In some control embodiments, the augmentation torque is adapted in real time under computer control based on muscle-tendon force (or muscle-tendon forces) or their corresponding biological joint torque (or torques) using different optimization functionals. For example, augmentation torque could be modulated by a control system so as to keep peak muscle-tendon force, or its time rate of change, below a threshold in order to reduce metabolic cost, or to lower the risk of musculoskeletal injury.

In some embodiments, a real-time estimate of muscle contractile element (CE) mechanical power, or its corresponding biological human joint power, serves as a controller input for a closed-loop torque control system. Such a direct, real-time neuromuscular control between biological CE power and wearable robotic applied torque enables an intuitive and volitional control without the need for complex and slow optimization routines. In various embodiments, aspects of inventive concepts herein involve a wearable robotic control strategy that uses one or more muscle-tendon interfaces to estimate one or more muscle CE powers, or their corresponding biological joint powers. Here a CE mechanical power applied about a biological joint is used to compute an augmentation torque for a robotic joint in parallel with that joint. A torque controller then servos to the augmentation torque target to directly control the output torque of the wearable robotic joint (see FIG. 1). In some example embodiments, a muscle-tendon interface presented herein uses real-time measurements to estimate muscle-tendon signals. Two or more elements of the muscle-tendon signal estimates are then used to compute muscle CE power, or its resulting biological joint power contribution. For each muscle-tendon interface, the muscle CE power is computed as the product of CE force and CE velocity. For each muscle spanning a joint, the CE power about the biological joint can be computed as that muscle's CE power multiplied by the product of the muscle's effective moment arm about the joint and the joint's rotational velocity, divided by the total muscle-tendon unit velocity. An exoskeleton augmentation power (the product of augmentation torque and joint rotational velocity) is then estimated from the muscle CE power about that same biological joint, and then used as a control target by an exoskeletal controller to modulate the corresponding device joint torque in parallel with that biological joint. In some control embodiments, the augmentation torque is functionally related to the biological CE muscle power, or powers, delivered to the biological joint that is being augmented by the parallel exoskeleton, divided by that joint's rotational velocity, using a linear or nonlinear function. In a some control embodiments, the augmentation torque is calculated based on different optimization functionals (e.g., such as minimizing muscle CE power).

In a some embodiments, a direct measurement of one or more muscle CE metabolic costs serves as controller command targets for a closed-loop torque control system. Such a direct neuromuscular control between muscle metabolic cost and wearable robotic torque delivers an intuitive and volitional control response without the need for complex and slow optimization routines. We present a wearable robotic control strategy that uses one or more muscle-tendon interfaces to estimate one or more muscle metabolic costs. The at least one muscle metabolic costs are used to compute an at least one augmentation torque. A torque controller then servos to the at least one augmentation torque target to directly control the at least one output torque of the wearable robot (see FIG. 1). For a given muscle, the metabolic rate depends on the muscle maximum isometric force (Fₘₐₓ), length where active muscle force is maximal (lₒₚₜ), the percentage of fast-twitch muscle fiber (FT), neural excitation, muscle activation, CE length, CE velocity, CE force, and CE isometric force. These biophysical parameters are then used to estimate muscle CE metabolism using the framework presented in [14]-[15]. An augmentation torque is then estimated from the real-time estimate of muscle metabolism, and then used as a control target by an exoskeletal controller to modulate the corresponding device joint torque in parallel with the biological joint. In some control embodiments, the augmentation torque is functionally related to the muscle metabolic cost, or metabolic rate, linearly or nonlinearly. In a second control embodiment, the augmentation torque profile is adapted by determining a profile that minimizes (or maximizes) an optimization functional. For example, a torque profile can be determined that minimizes muscle metabolic cost for at least one muscle.

In a some embodiments, a direct measurement of one or more muscle efficiencies serves as controller command targets for a closed-loop torque control system. In such embodiments, muscle efficiency is defined as the CE mechanical power divided by the CE metabolic power. In various embodiments, aspects of inventive concepts herein in involve a wearable robotic control strategy that uses one or more muscle-tendon interfaces to estimate one or more muscle efficiencies. The at least one muscle efficiency is used to compute an at least one augmentation torque. A torque controller then servos to the at least one augmentation torque target to directly control the at least one output torque of the wearable robot (see FIG. 1). For a given muscle, the efficiency depends on the muscle maximum isometric force (Fₘₐₓ), length where active muscle force is maximal (lₒₚₜ), the percentage of fast-twitch muscle fiber (FT), neural excitation, muscle activation, CE length, CE velocity, CE force, and CE isometric force. These biophysical parameters are then used to estimate muscle CE efficiency using the framework presented in [14]-[15] for metabolic power, and CE velocity multiplied by CE force for CE mechanical power. An augmentation torque is then estimated from the real-time estimate of muscle efficiency, and then used as a control target by an exoskeletal controller to modulate the corresponding device joint torque in parallel with the biological joint. In one control embodiment, the augmentation torque applied to a joint is functionally related to the efficiencies of the muscles spanning that joint, linearly or nonlinearly. In a some control embodiments, the augmentation torque modulation is adapted based on different optimization functionals (e.g., such as maximizing muscle efficiency for at least one muscle). In some control embodiments, the augmentation torque modulation profile is adapted to enable at least one muscle to operate in a favorable CE velocity range, wherein muscle efficiency is maximized. For skeletal muscle, efficiency is typically maximized in the 0.15 to 0.3 V/Vmax range [14]-[15]. Hence, from a real-time estimate of CE velocity, and an a priori estimate of Vmax, the augmentation torque profile is modulated to keep the V/Vmax ratio within this optimal range of values so as to optimize efficiency. Since muscle power is also maximized within a similar V/Vmax range, this controller would maximize power delivery and efficiency. This control approach is comparable to how bicycle gears are used to enable the cyclist to operate at maximal efficiency and power as the road continually changes inclination.

Several control architectures are described herein, including power steering, augmentation objective-based control, and neuro-reflexive control.

Aspects of inventive concepts are generally directed to a robotic control system that may comprise a wearable robot comprising at least one actuated joint; at least one muscle-tendon interface configured to measure at least one physiological signal; a torque set point processor configured to receive the at least one physiological signal from the at least one muscle tendon interface and estimate a muscle-tendon state corresponding to a human motor intention and to compute at least one augmentation joint torque based on the muscle-tendon state; and a torque controller configured to apply the at least one augmentation joint torque to the at least one actuated joint of the wearable robot.

In various embodiments, the torque set point processor may comprise a human motor intention estimator configured to receive the at least one physiological signal from the at least one muscle-tendon interface and estimate the muscle-tendon state; and an augmentation strategy module configured to receive the estimate of the muscle-tendon state and compute the at least one augmentation joint torque.

In various embodiments, the at least one muscle-tendon interface may comprise at least two markers and at least one sensor and the human motor intention estimator comprises a marker tracking module configured receive information from the at least one sensor and measure the distance between the at least two markers at a tendon to estimate a tendon elongation.

In various embodiments, the human motor intention estimator may be configured to use the tendon elongation and an estimated or measured tendon stiffness value to estimate a muscle-tendon force.

In various embodiments, the at least one muscle-tendon interface may comprise at least two markers and at least one sensor and a marker tracking module configured to receive information from the at least one sensor and measure a distance between the at least two markers at a muscle and estimate the time rate of change of the distance.

In various embodiments, the human motor intention estimator may comprise a feature extraction module configured to receive information from the marker tracking module and estimate a muscle or tendon vibration.

In various embodiments, the human motor intention estimator may be configured to use the tendon vibration and a length of the tendon to estimate a muscle-tendon force.

In various embodiments, the human motor intention estimator may comprise a muscle-tendon model module configured to receive information from the marker tracking module and the feature extraction module and estimate a muscle-tendon force.

In various embodiments, the at least one muscle-tendon interface may comprise at least one sensor and the human motor intention estimator may comprise a tissue elastography module configured to receive information from the at least one sensor.

In various embodiments, the at least one sensor may be configured to pulse a muscle or tendon and the tissue elastography module may be configured to estimate the muscle or tendon stiffness based, at least in part, on the muscle or tendon response to the pulse as detected at the at least one sensor.

In various embodiments, the human motor intention estimator may be configured to use the muscle or tendon stiffness to estimate a muscle-tendon force.

In various embodiments, the human motor intention estimator may comprise an image processing module configured to estimate at least one of muscle length, muscle velocity, muscle speed, or muscle volume.

In various embodiments, the at least one muscle-tendon interface may comprise at least one pressure/force sensor configured to be positioned between the skin surface adjacent the muscle and an elastic sleeve that wraps around the limb circumferentially and may be configured to detect at least one of muscle contraction, volumetric changes, or stiffness changes.

In various embodiments, the human motor intention estimator may comprise a feature extraction module configured to receive information from the at least one pressure/force sensor or the at least one EMG sensor and process raw signals into muscle activity measurements.

In various embodiments, the at least one muscle-tendon interface may comprise at least one EMG sensor constructed and arranged to be positioned on the skin surface and configured to collect electric muscle activity adjacent a muscle.

In various embodiments, the human motor intention estimator may comprise a muscle-tendon model module configured to receive information from at least one of the image processing modules or the feature extraction module and output a muscle-tendon force.

In various embodiments, the human motor intention estimator may comprise a joint segment geometry module configured to receive information from the muscle-tendon model module and estimate a human torque for the target joint.

In various embodiments, the at least one muscle-tendon interface may comprise a first ultrasound generator configured to emit ultrasound waves toward a target at a first frequency; a second ultrasound generator configured to emit ultrasound waves toward the target at a second frequency; and an ultrasound receiver configured to receive ultrasound wave reflected from the target, wherein the first frequency and the second frequency are different.

In various embodiments, the image processing module may be configured to receive a signal from the ultrasound receiver corresponding to the ultrasound wave reflected from the target and calculate a muscle-tendon velocity or a muscle-tendon speed.

In various embodiments, an acoustic lens may be coupled to the ultrasound receiver.

In various embodiments, the augmentation strategy module may compute the at least one augmentation joint torque in real time.

In various embodiments, the augmentation strategy module may adjust the at least one augmentation joint torque monotonically in accordance with corresponding changes in the estimate of the muscle-tendon state.

In various embodiments, the augmentation strategy module may adjust the at least one augmentation joint torque to keep peak muscle-tendon force below a threshold to reduce metabolic cost.

In various embodiments, the augmentation strategy module may adjust the at least one augmentation joint torque to optimize at least one of maximizing muscle efficiency for at least one muscle or minimizing muscle metabolic rate.

In various embodiments, the torque controller may be configured to apply the at least one augmentation joint torque to the at least one actuated joint of the wearable robot within 100 ms of the at least one muscle-tendon interface measuring the at least one physiological signal.

Aspects of inventive concepts are generally directed to a method to control at least one actuated joint of a wearable robot. The method may comprise measuring at least one physiological signal using at least one muscle-tendon interface; estimating a muscle-tendon state corresponding to a human motor intention; computing at least one augmentation joint torque based on the muscle-tendon state; and applying the at least one augmentation joint torque to the at least one actuated joint of the wearable robot.

In various embodiments, the method may comprise applying the at least one augmentation joint torque to the at least one actuated joint of the wearable robot within 100 ms of measuring the at least one physiological signal using at least one muscle-tendon interface.

In various embodiments, the method may comprise estimating muscle length or muscle speed from an ultrasound image using an affine transformation.

In various embodiments, the method may comprise estimating muscle length or muscle speed from an ultrasound image using an affine transformation, a neural network, and a stochastic filter.

In various embodiments, the at least one physiological signal may comprise at least one of muscle displacement, tendon displacement, muscle-tendon force, tendon wave speed, muscle stiffness, muscle electromyography, muscle velocity, muscle speed, tendon velocity, tendon speed, muscle-tendon unit (MTU) length, joint rotational position, rotational velocity, or rotational acceleration.

In various embodiments, the muscle-tendon state may comprise at least one of a muscle contractile element (CE) velocity, a muscle contractile element speed, a muscle-tendon unit (MTU) velocity, a muscle-tendon unit (MTU) speed, a human biological muscle force, a biological joint torque, a biological CE mechanical power, muscle metabolic power, muscle efficiency, or muscle fatigue.

In various embodiments, the image processing module may be configured to apply a Hilbert transform to the signal from the ultrasound receiver to generate an envelope of the received ultrasound signal.

In various embodiments, the image processing module may be configured to apply a bandpass filter to the envelope of the received ultrasound signal.

In various embodiments, the image processing module may be configured to apply a wavelet transform to the band-passed envelope of the received ultrasound signal with a Morlet kernel.

In various embodiments, the image processing module may be configured to find a frequency with the largest energy and calculate a muscle-tendon velocity or a muscle-tendon speed.

In various embodiments, the at least one of the first ultrasound generator or the second ultrasound generator may be coupled to an acoustic holographic layer.

In various embodiments, the at least one of the first ultrasound generator or the second ultrasound generator may be coupled to a gel pad.

In various embodiments, the wearable robot may comprise an actuator coupled to the at least one actuated joint and the torque controller may be configured to apply the at least one augmentation joint torque to the actuator.

In various embodiments, the torque controller may operate under closed-loop control.

In various embodiments, the muscle-tendon state may comprise mechanical dynamics or at least one energetic estimate of a muscle-tendon unit.

In various embodiments, the mechanical dynamics may comprise at least one of a muscle contractile element (CE) velocity, a muscle contractile element speed, a muscle-tendon unit (MTU) velocity, a muscle-tendon unit speed, a human biological muscle force, a biological joint torque, or a biological CE mechanical power.

In various embodiments, the at least one energetic estimate may comprise at least one of muscle metabolic power, muscle efficiency, or muscle fatigue.

In various embodiments, the image processing module may be configured to estimate muscle length, muscle velocity, or muscle speed from an ultrasound image using an affine transformation.

In various embodiments, the image processing module may be configured to estimate muscle length, muscle velocity, or muscle speed from an ultrasound image using an affine transformation, a neural network, and a stochastic filter.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The foregoing will be apparent from the following more particular description of example embodiments, as illustrated in the accompanying drawings in which like reference characters refer to the same parts throughout the different views. The drawings are not necessarily to scale, emphasis instead being placed upon illustrating embodiments.

FIG. 1 shows different example embodiments of a robotic control system, in accordance with aspects of inventive concepts.

FIG. 2 shows different example embodiments for using ultrasound or magnetic bead tracking at a robotic control system, in accordance with aspects of inventive concepts.

FIG. 3 shows different example embodiments for using non-invasive techniques at a robotic control system, in accordance with aspects of inventive concepts.

FIG. 4A and FIG. 4B show an example embodiment of a first ultrasound generator, a second ultrasound generator, an ultrasound receiver, and an interference pattern, in accordance with aspects of inventive concepts.

FIGS. 5A-5F show example embodiments of aspects of a non-invasive ultrasound Doppler velocimetry signal processing pipeline, in accordance with aspects of inventive concepts.

FIG. 6A shows an example embodiment of a first ultrasound generator, a second ultrasound generator, an ultrasound receiver, and a layer of ultrasound transparent material, in accordance with aspects of inventive concepts.

FIG. 6B shows an example embodiment of a first ultrasound generator, a second ultrasound generator, an ultrasound receiver, a layer of skin and fat, a layer of muscle and tendon, and a layer of bone, in accordance with aspects of inventive concepts.

FIG. 7A shows an example embodiment of an ultrasound generator with acoustic holographic layer on top of it, in accordance with aspects of inventive concepts.

FIG. 7B shows an example embodiment of an ultrasound generator coupled to an acoustic holographic layer and a gel pad, in accordance with aspects of inventive concepts.

FIG. 8A shows an example embodiment of interference fringes movement along with muscle/tendon movement, in accordance with aspects of inventive concepts.

FIG. 8B shows an example embodiment of interference fringes, an ultrasound receiver, and an acoustic lens system, in accordance with aspects of inventive concepts.

FIG. 9 shows example embodiments of non-invasive acoustic, strain, and impulse generator sensing and actuation systems, devices, and methods, in accordance with aspects of inventive concepts.

FIG. 10 shows example embodiments of non-invasive kinetic and kinematic sensing systems, devices, and methods, in accordance with aspects of inventive concepts.

FIG. 11 shows an example embodiment of a control architecture for power steering control, in accordance with aspects of inventive concepts.

FIG. 12 shows an example embodiment of a control architecture for augmentation objective-based control, in accordance with aspects of inventive concepts.

FIG. 13 shows example objective functions for augmentation strategies, in accordance with aspects of inventive concepts.

FIG. 14 shows an example embodiment of a control architecture for reflexive control, in accordance with aspects of inventive concepts.

FIG. 15 shows an example embodiment of a control architecture for reflexive and volitional control, in accordance with aspects of inventive concepts.

FIG. 16 shows an example embodiment of a control architecture for reflexive control using force feedback, in accordance with aspects of inventive concepts.

FIG. 17 shows two example ultrasound images from the probe at the same position after the affine transform, in accordance with aspects of inventive concepts.

FIG. 18 shows an example overall system diagram for the noninvasive ultrasound imaging pipeline to estimate muscle length, muscle speed, or muscle velocity, in accordance with aspects of inventive concepts.

FIG. 19 shows the image quality optimizer from the FIG. 18 diagram, in accordance with aspects of inventive concepts.

FIG. 20 shows the pipeline for the muscle length estimator from the FIG. 18 diagram, in accordance with aspects of inventive concepts.

FIG. 21 shows an example of the SIFT (Scale-Invariant Feature Transform) matching between two ultrasound frames, in accordance with aspects of inventive concepts.

### DETAILED DESCRIPTION

A description of example embodiments follows.

This document presents muscle-tendon interfaces and control strategies for wearable robotic systems that are used in human augmentation, rehabilitation, and permanent assistance. It is to be understood that the domain of wearable robots as used herein extends to both therapeutic and augmentation applications including orthotic, prosthetic and exoskeletal devices. In the proposed framework, the wearable robot has at least one actuated joint that includes at least one muscle-tendon interface, with its corresponding biophysical analyses, for the real-time estimation of a biomechanical and/or physiological parameter such as biological human joint torque, muscle contractile power, muscle metabolic power and/or muscle efficiency, among others, to inform one or more augmentation torque commands to be sent to the wearable robotic control system. A control framework is presented wherein a powered exoskeleton applies closed-loop (servo) control of the one or more augmentation torques under computer control about the one or more exoskeletal joints.

In some embodiments, each exoskeletal augmentation torque is functionally related to its corresponding biological human joint torque. In other embodiments, each exoskeletal augmentation torque is functionally related to its corresponding biological muscular contractile element (CE) power. In a other embodiments, each exoskeletal augmentation torque is functionally related to its corresponding biological muscular metabolic power. In a power-steering framework, each augmentation joint torque monotonically increases/decreases in real time with real time increases/decreases in its corresponding biological muscle torque, muscle CE power, or muscle metabolic power using either a linear or non-linear functional relationship.

Several embodiments of muscle-tendon interfaces, and their biophysical analyses, are presented to estimate in real time using an autonomous wearable system muscle-tendon biomechanical and physiological parameters, including muscle-tendon force, tendon stretch, tendon velocity, tendon stiffness, muscle length, muscle velocity, muscle stiffness, muscle-tendon moment arm(s), and/or the associated derivatives of these, all in relation to a rotational joint axis, or axes. A biophysical analysis then translates these signals to a real-time estimate of muscle-tendon state, a vector comprising a real-time estimate of one or more biomechanical/physiological parameters including at least one of a muscle contractile element (CE) velocity, muscle-tendon unit (MTU) velocity, human biological muscle force, biological joint torque, biological CE mechanical power, muscle metabolic power, muscle efficiency, or muscle fatigue. These methods include wearable ultrasound systems, an acoustic sensory method, mechanomyography, electromyography, and an implanted magnetic bead tracking system called magnetomicrometry. Additionally, wearable sensors such as inertial measurement units (IMUs) and pressure insoles are described for the estimation of muscle and tendon dynamics.

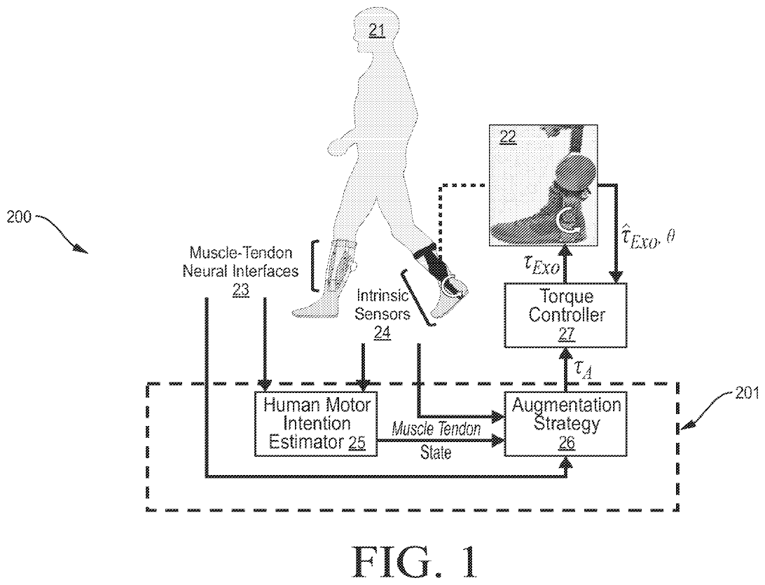

As shown in FIG. 1, the exoskeletal control system comprises several computational modules including a Human Motor Intention Estimator, Augmentation Strategy, and a joint Torque Controller. The Human Motor Intention Estimator receives information from at least one muscle-tendon interface, and potentially other intrinsic wearable sensors, to estimate muscle-tendon state. The real-time estimates of muscle-tendon state of the at least one muscle-tendon unit are then inputted into an Augmentation Strategy module that computes in real time at least one augmentation joint torque, or computationally adapts across multiple gait cycles towards at least one optimal torque profile. A Torque Controller module then applies a closed-loop control, “servoing” at least one wearable robotic actuator to apply at least one augmentation torque about at least one robotic joint. Here, we define closed-loop control to mean the application of feedback and dynamic feedforward compensation—including inverse dynamic compensation—to make the applied exoskeletal torque follow an augmentation torque command target. Without loss of generality, herein we will interchangeably refer to “closed-loop” control as “servoing” an output to follow a target.

It should be understood that the augmentation torque can be computed as a function of joint equilibrium angle and impedance terms, and these terms can be adapted in real time to modulate augmentation torque in an updating manner. Here, the impedance about the joint equilibrium can form at least one of a linear or non-linear functional relationship with the joint displacement derivatives. In the embodiments herein, the term “augmentation torque” refers to an exoskeletal torque that is computed explicitly, or is implicitly defined as a time-varying function of joint equilibrium and impedance, or both. Here impedance refers to a linear or non-linear relationship between the augmentation torque, or force, contribution and the joint, or muscle, displacement, and its derivatives. A spring or damper are examples of impedance contributions to an augmentation torque. Equilibrium refers to a static displacement at which no augmentation torque, or force, is applied.

### Wearable Robots

In some example embodiments, a wearable robot, such as an exoskeleton, can comprise a powered actuator attached in some manner to the human body. The exoskeleton can be affecting one or multiple joints, including but not limited to the ankle, knee, hip, back, wrist, elbow and shoulder. In some example embodiments, the torque controller of the exoskeleton receives an input from the neuromuscular interface and computational system, and outputs torque to achieve the desired augmentation goal. The torque from the exoskeleton can be used to achieve a desired joint position or impedance.

In some example embodiments, one or multiple muscle-tendon interfaces can act as the input to the exoskeleton controller. These interfaces act to decode user intent for the purpose of controlling the exoskeleton. The muscle-tendon interfaces are meant to be non-invasive or minimally invasive, as well as high bandwidth for real time control of the exoskeleton. In the next section, several embodiments of muscle-tendon interfaces are described in detail each with distinct advantages and disadvantage.

### Muscle-Tendon Interfaces

A muscle-tendon neural interface may also be referred to as a muscle-tendon interface, a muscle-tendon sensor, or a sensor herein. A muscle-tendon interface can be an interface at one or more muscle, an interface at one or more tendon, or an interface at one or more muscle and one or more tendon.

### Ultrasound

In some example embodiments, a muscle-tendon interface comprises a wearable ultrasound system that has a thin, stretchable probe and can capture continuous B-mode or M-mode images in real time. The wearable ultrasound system can have a frequency and depth that target muscle-tendon structures. The ultrasound system can be arranged in either a 1D or 2D array configuration. In some example embodiments, the wearable ultrasound system is composed of piezoelectric components that can operate at different frequencies compatible with ultrasound imaging. These piezoelectric components are embedded within a flexible material that can conform to the shape of the biological body upon which it is being placed, and are arranged in a grid pattern, in either a row or column, or combination of both. The transducers are commonly arranged in a phased array configuration. The transducers are all individually connected, allowing them to be powered and send information back to a wearable computer for imaging of the raw RF data. The wearable ultrasound probe can be connected to a beamformer in which the radiofrequency signals are processed into a 2D or 3D image. In some example embodiments, post-processing is performed on wearable electronic boards or sent wirelessly to a dedicated remote computer to complete post-processing steps. The wearable ultrasound system may be attached to the body either through a fabric, adhesive, or any other means to ensure a uniform pressure and contact. The ultrasound system can be used with or without gel, gel being able to amplify the given image. The ultrasound system can capture information across a wide range of frames per second, such as from 10-120 frames per second.

Image Processing from Ultrasound

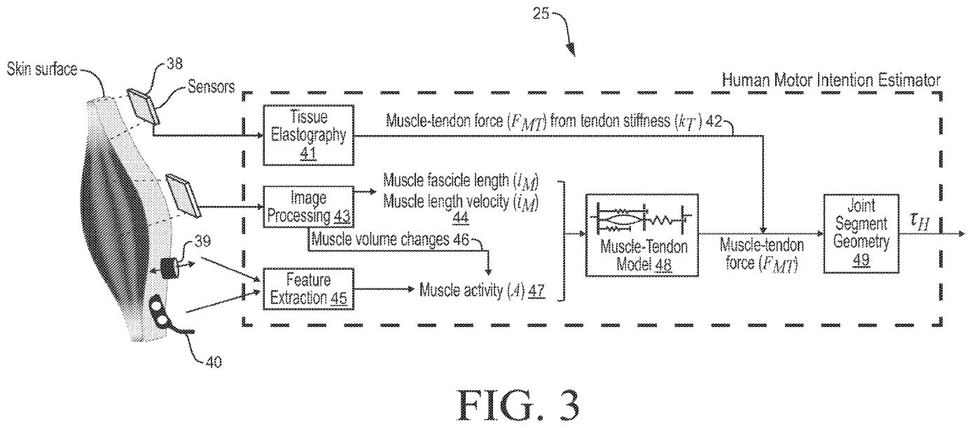

In some embodiments, an ultrasound probe is placed above the muscles and tendons of interest over the skin to track tissue dynamics, such as length and velocity in real-time (see FIG. 3). Using a single ultrasound probe, an image processing algorithm can be used to extract the tissue features from the output B-mode images, such as fascicle changes of the muscle to provide a direct measurement of muscle length and velocity. The same process may be applied to the tendon to extract tissue features. Features may include but are not limited to muscle aponeuroses, muscle fascicle direction, pennation angle and length, muscle thickness and depth, tendon length, or tendon shape, such as cross-sectional area. These features can be captured across time, allowing transient and steady-state changes to be tracked. In some example embodiments, the image processing may include, but is not limited to, optical flow algorithms, edge detection, filtering, denoising or resampling. The image processing analyzes the ultrasound B-mode frames and outputs tissue features in real-time. When using either a 2D ultrasound array or more than one ultrasound probe, the image processing algorithm can be used to generate a 3D image of the tissue. This can be used to compute the volumetric changes of the muscle, giving a predictor of muscle activity. In another example, neural networks can be used to reduce latency in image processing techniques based on training data collected previously. The machine learning approaches may include, but are not limited to, linear regression or convolutional neural networks. This approach enables a precise and rapid identification of tissue features and enhances feature tracking, enabling real-time tissue information for various tasks.

In alternative embodiments, the ultrasound system can track vibrations occurring in the muscle or tendon during dynamic tasks. Image processing for tracking vibrations includes but is not limited to speckle tracking. The natural and resonant vibrations of the muscle can be used to estimate muscle activation. The same vibrations occurring in the tendon may be measured to estimate muscle force. This tendon vibration approach can be accomplished using the equation

$$2 fl = \sqrt{\frac{σ}{ρ}} ,$$

where f is the vibrational frequency, l is the length of the medium observed (tendon length for example), and σ is the axial stress, while p is the density of the medium. Therefore, if you know vibrational frequency, the force can be calculated as F=(2fl)²*ρA, where A is the tendon cross-sectional area estimated using ultrasound data, l is the tendon length approximated also from ultrasound data, and ρ is tendon density taken from the literature.

In another example, rather than capturing the resonant frequency, wave propagation speeds can be induced by an acoustic or mechanical impulse on the tissue, such as through focused ultrasound beams, or by an external actuated pulse. The following methods comprise a shear wave model that calculates force. The force calculation is based, at least in part, on frequency or wave speed of the tissue. Measuring the induced shear wave through ultrasound imaging can both provide an estimate on the stiffness and elastic properties k of the tissue, and also provide the wave speed occurring in the tissue, leading to an estimated measurement of muscle-tendon force using the equation

$$c = 2 fl = \sqrt{\frac{σ}{ρ}} ,$$

where c is the wave speed through the tissue, or F=(c)²*ρA.

### Ultrasound-Based Marker Tracking

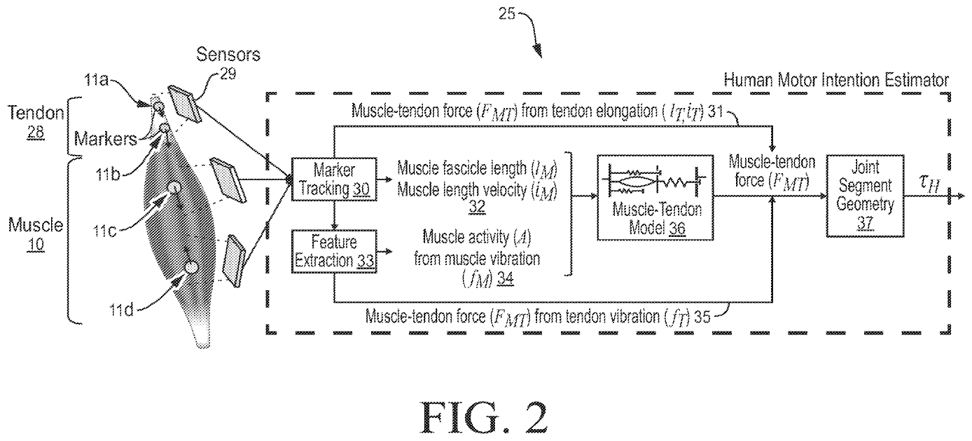

In some embodiments, an ultrasound probe can track an implanted marker, or markers, and use image processing to infer dynamics of the marker(s) moving within the tissue to estimate tissue dynamics through ultrasound imaging (see FIG. 2). The implanted markers can be biocompatible such that they can be implanted in tissue with little to no risk. The materials of the implant may include, but are not limited to, titanium, stainless steel, tantalum, hydrogel or other material that may show up with high contrast on ultrasound imaging while also having biocompatible properties. A marker can have a significant acoustic impedance difference compared to the tissue it is implanted within in order to generate large contrast differences. Other materials include those which are tuned to match the tissue impedance (stiffness/damping) properties of the surrounding implant tissues. For example, a hydrogel marker on the tendon would have similar mechanical properties to the tendon in terms of elasticity and improve biocompatibility and comfort. The marker, or markers, are either used as reference points within the tissue for calibration procedures, or as points to actively track through dynamic changes in the muscle and/or tendon tissues. Marker tracking involves, but is not limited to, contrast thresholding to isolate the marker and only track the marker as it moves with the tissue. The distance it moves can be tracked by analyzing the pixel distance it moves across the ultrasound image. In some instances, with one ultrasound probe, the 2D position of the marker can be tracked according to the plane the ultrasound probe is oriented to. The transient changes of the marker can also be tracked as the probe collects data continuously and is subsequently analyzed. With multiple ultrasound probes or a 2D array, the 3D position of the marker may be measured through localization algorithms in reference to the location of the ultrasound probes or arrays to one another. Ultrasound probes, or arrays, aligned across the skin surface in alignment with the muscle or tendon can be used to track one or more implanted markers within the tissue. This method is important to ensure the marker can be tracked even if it falls out of the plane of one of the ultrasound probes, or arrays. For example, the change in distance between two markers placed at each end of the tissue, measured through the ultrasound probes, or arrays, can be used to compute length change in the tissue. Given the change in distance of the two markers in a set time period, the velocity of the tissue can be calculated. There can be one or more implanted markers across the tissue length. The markers can be positioned in any way across the tissue, such as across the fascicle direction or at the proximal and distal ends of the muscle. The markers can be any shape or size, including but not limited to spheres, sheets or fibers. Some shapes, such as coils, nets or other structures may be used to promote integration with the tissue to prevent migration in dynamic tissue such as muscle or tendon.

Ultrasound-based tracking can be used to indirectly measure tendon force through tendon shear wave and tendon transverse frequency measurements. In some embodiments, an ultrasound marker is placed in the tendon, such as a sheet or small bead. When a force is applied to the tendon, the tendon's transverse vibrations can be tracked through the ultrasound patch measuring the vibrations through displacements of the marker, serving either as a reference point or as a point that is actively tracked. As described earlier, such a measured transverse frequency [symbol] of the tendon can used to calculate tendon force, or F=(2fl)²*ρA, where A is the tendon cross-sectional area estimated using ultrasound data, l is the tendon length approximated also from ultrasound data, and ρ is tendon density taken from the literature.

In some embodiments, an ultrasound marker in the shape of a fiber or sheet can be implanted onto the tendon's surface, and the relative elongation in the tendon can be measured indirectly through the strain in the marker detected by the ultrasound patch. Tendon elongation multiplied by tendon modulus, estimated by the ultrasound-measured cross section of the tendon, can be used to estimate tendon force. Alternatively, two marker beads can be implanted into the tendon along its longitudinal axis, and the distance between the markers can be estimated in real time using the ultrasound imaging probe, or array, as a correlate of tendon elongation. This tendon elongation when coupled with an estimated tendon stiffness value correlates to muscle-tendon force.

In some embodiments, given an induced mechanical stimulus across the tendon, the shear wave can be measured using the disturbance in the markers from the stimulus. More than one marker may be placed either along or across the tendon, this enables the measurement of the wave speed difference between two or more markers. Tendon force can then be estimated using the equation F=(c)²*ρA, where A is the tendon cross-sectional area estimated using ultrasound data, and ρ is tendon density taken from the literature.

### Tissue Doppler (M-Mode)

In some example embodiments, With the ultrasound probes or arrays, tissue Doppler of a 2D image can be utilized to extract tissue velocity and strain rate (see FIGS. 2 and 3). Doppler can be utilized in cases where image processing cannot achieve the required latency necessary for control or to track quick, transient changes in the muscle-tendon dynamics. The Doppler can be tuned to focus on phase and frequency shifts specific in muscle and tendon tissues. The Doppler can also be used in combination with the implanted markers to extract marker velocity.

### Tissue Elastography

In some example embodiments, with the ultrasound probes, tissue elastography can be used to estimate the stiffness of the tissue (see FIG. 3). The stiffness of the muscle or tendon can be an estimator for muscle force and activation. The tissue elastography pulse can be induced by either an actuated shear wave on the outside of the skin or through an acoustic radiation force impulse from the ultrasound system. In one example, tissue elastography can estimate the stiffness of the tendon, and using a non-linear mapping, can correlate to muscle-tendon force.

### A-Mode

In some example embodiments, an A-mode configuration with the ultrasound probes, or arrays, includes transmitters and receivers oriented across the tissue of interest. Based on the time of flight from the transmitter to receiver, this can indicate the changes in tissue thickness and volume, relating to muscle activation measurements.

### Ultrasound Doppler Velocimetry

In some example embodiments, aspects of inventive concepts herein involve an Ultrasound Doppler Velocimetry device designed to measure the movement speed of a muscle and tendon noninvasively. In some example embodiments, the device comprises two ultrasound generators and one ultrasound receiver.

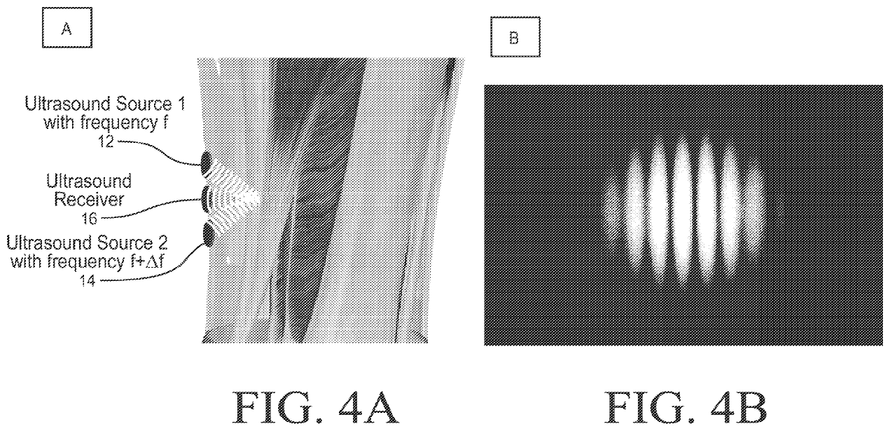

The two ultrasound generators generate two ultrasound beams with slightly different frequencies that are focused at the same point on the surface of the muscle or tendon that is being imaged, as shown in FIG. 4A. At the focusing point, a moving bar shape interference pattern will be created by the two ultrasound beams, as shown in FIG. 4B. The bar moving velocity is determined by the frequency difference between the two ultrasound beams.

The ultrasound receiver detects the reflected sound wave from the muscle or tendon surface that is being imaged. The intensity of the detected signal will change periodically, and its frequency is correlated with the muscle or tendon moving velocity. The frequency can be easily calculated by performing a Fourier transform upon the received ultrasound signal intensity. The muscle-tendon velocity can be calculated using the following method. Assume ultrasound frequencies from the generators are [symbol] and [symbol] where [symbol] . Since the interference region is quite small, backscatters in the region move at the same speed [symbol] . The wavelength is calculated as

$$λ = \frac{V}{f} ,$$

where the ultrasound speed in muscle-tendon is [symbol] . Since the ultrasound interference fringes periodically go through the backscatters, the frequency of the energy of the backscattered ultrasound from a single backscatter is

$${f}_{d} = \frac{v}{Δ s} + Δ f$$

where the distance between fringes is

$$Δ s = \frac{λ}{2 \cdot sin ( ϕ )} .$$

Since all the backscatters move at the same speed, the reflected ultrasound signals' energy will change at the same frequency [symbol] . Finally, the muscle/tendon movement speed [symbol] can be detected by finding [symbol] .

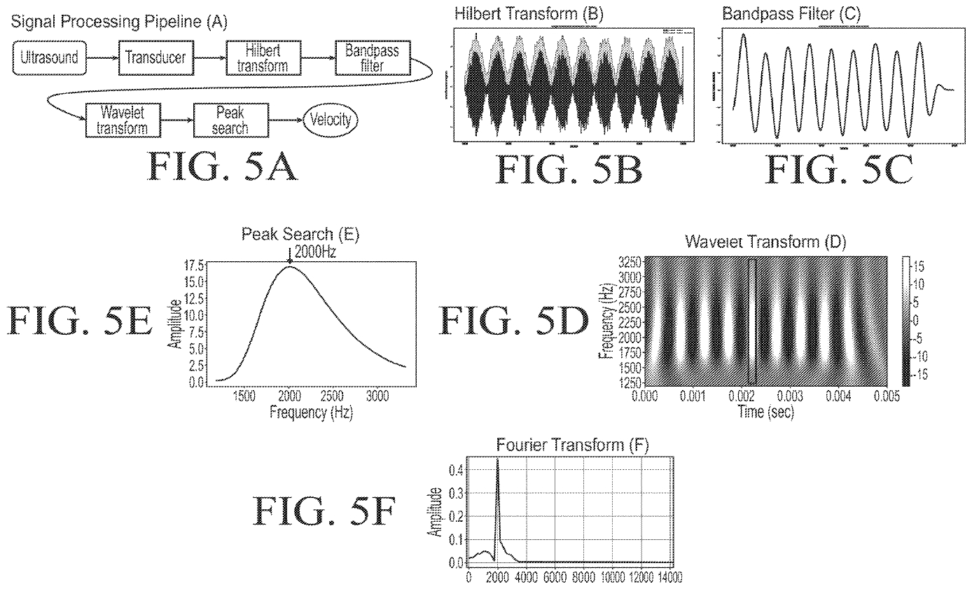

Presented herein is an example signal-processing pipeline to calculate either the muscle or tendon velocity [symbol] from the received ultrasound signal [symbol] . The schematic is shown in FIG. 5A through 5F. First, the ultrasound signals on the receiver transducer are converted to voltages. Then the voltages go through Hilbert transform to get its envelope. The envelope's main frequency will be [symbol] . To detect the envelope's main frequency, we first apply a bandpass filter to only keep the possible frequencies which are estimated by

$$Δ f \pm \frac{{v}_{max}}{Δ s} .$$

After that, there are several methods to detect the filtered signal's main frequency. Those methods include but are not limited to:
1. Wavelet transform, then pick the frequency with the max energy.
2. FFT, then pick the frequency with the max energy.
3. Calculate the average time between zero crossing points, then calculate

$${F}_{d} = \frac{1}{2 T} .$$

Finally, muscle-tendon velocity [symbol] is determined by the equation

[symbol]

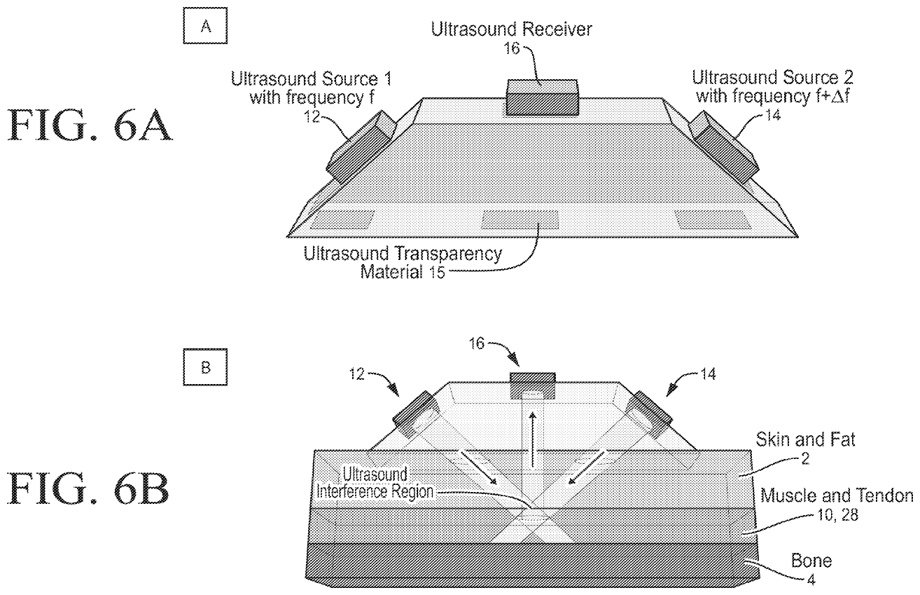

There is more than one way to implement Ultrasound Doppler Velocimetry. In this description we present two methods. The first method is shown in FIG. 6A where the two ultrasound generators are placed on an ultrasound transparency material at an angle. The distance and angle between the two ultrasound generators are determined by the depth of the target muscle or tendon that is being imaged. The two ultrasound beams should focus on the same point on the muscle or tendon surface, as is shown in FIG. 6B.

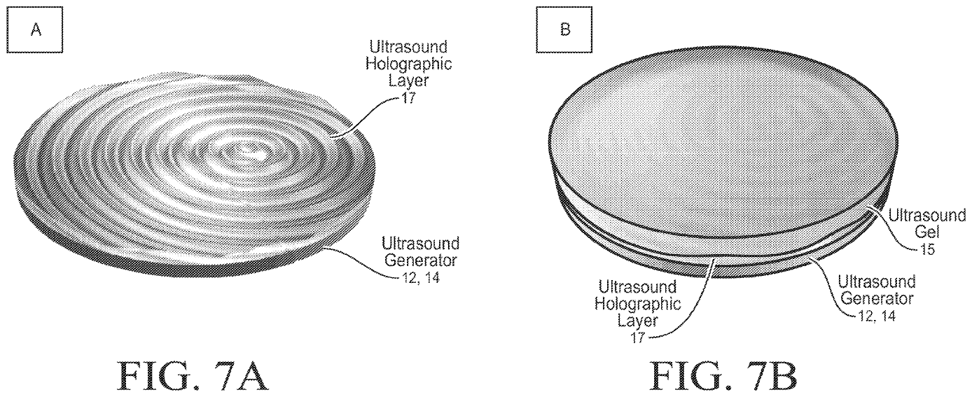

Another method is steering and focusing the ultrasound beam to the surface of the muscle or tendon being imaged with ultrasound holographic layers on each ultrasound generator, as is shown in FIG. 7A. All the sensors can be placed on the skin flatly with sticky gel pads, as shown in FIG. 7B by this method.

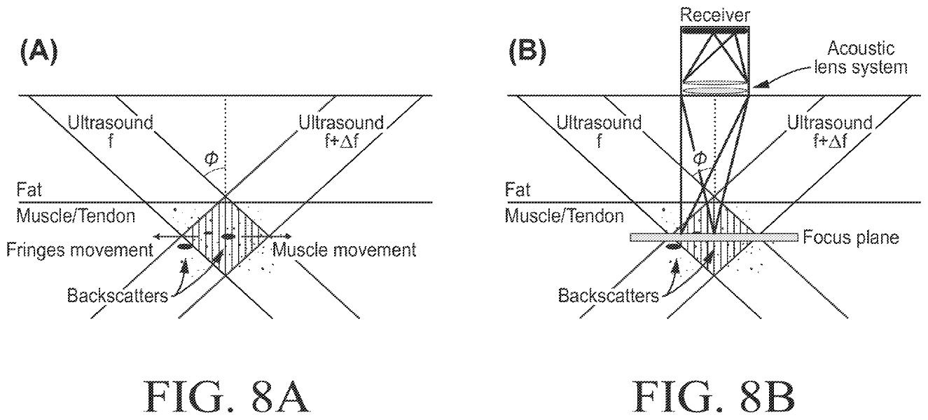

The receiver can also embody different designs to enhance the received signal from the interference patterns. In FIG. 8 for example, the receiver contains an acoustic lens system which ensures projection of the center plane of the interference area onto the receiver.

An important design attribute of this interface embodiment is that signal integrity is detectable since “zero muscle velocity” results in a structured, time-varying fringe pattern that is predictable based solely upon the frequency difference of the two transducers. Absence of this pattern signals a sensor fault. In an exoskeleton that uses this sensor interface, such fault detection serves as a risk control measure in support of international standards like ISO 14971 (Application of Risk Management to Medical Devices) and IEC 60601 (Medical Electrical Equipment—General Requirements for basic safety and essential performance).

### Noninvasive Ultrasound Imaging to Estimate Muscle Length and Velocity

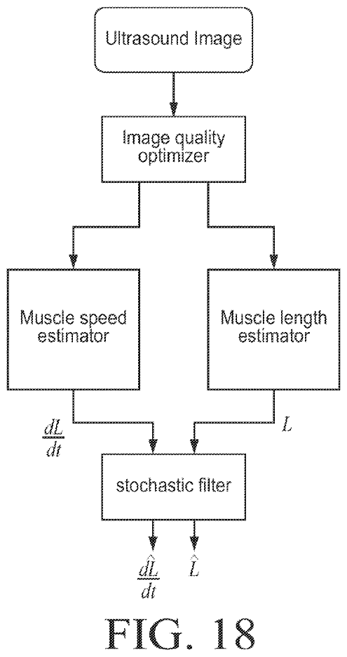

To calculate the length and velocity dynamics of a muscle using noninvasive ultrasound, the pipeline described herein is an example of an image processing module. In some embodiments, the method comprises multiple subsystem processes:
1. Image quality optimizer;
2. Muscle speed estimator;
3. Muscle length estimator; and
4. stochastic filter.
   The pipeline begins with the current captured ultrasound image which is input into the image quality optimizer. As described below, this applies basic filters and image adjustment to improve the muscle speed and length estimators. The processed image is then input into both muscle speed and length estimators, which output estimates or probability distributions of the muscle length and speed. These outputs are then processed into a stochastic filter, that improves the accuracy of estimation given noise and system characteristics. The diagram showing the pipeline to estimate muscle length and speed is shown in FIG. 18 comprising ultrasound image inputs, image quality optimizing, muscle speed and length estimators, and stochastic filtering. These modules and estimators are described in more detail below.

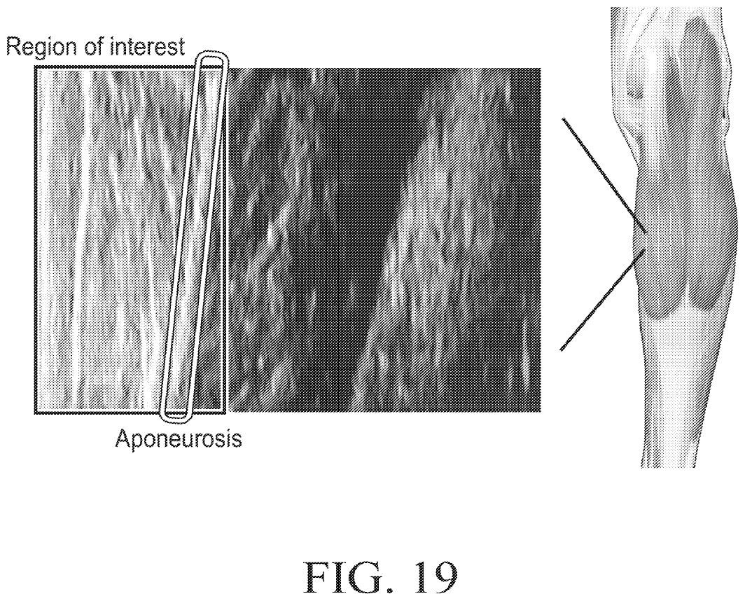

Image quality optimizer: The image quality optimizer, as shown in FIG. 19, performs the following operations to the ultrasound image:
1. Removing imaging noise;
2. Find and crop out the region of interest; and
3. Tuning the contrast of the ultrasound image.

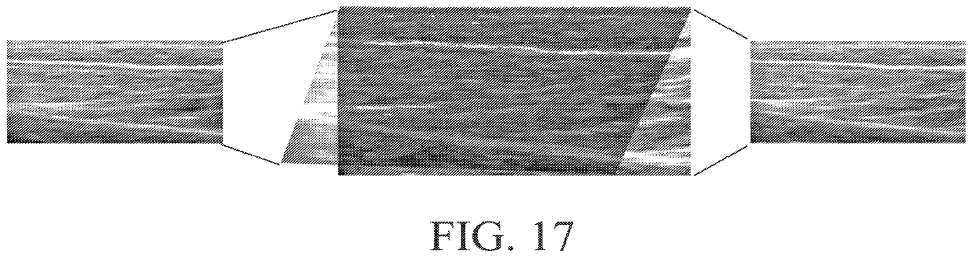

Muscle speed estimator: When a muscle contracts, we can see in FIG. 17 that ultrasound sequential images documenting the muscle's contraction match one another after performing an affine transformation. The muscle's length change and speed are found through the parameters of the affine transformation.

There are three types of skeletal muscle within the human body. For muscles whose fibers are parallel to one another, we will see translation and scaling in the fibers' direction in the ultrasound images during movement. For unipennate and bipennate muscles, we will see both translation and shearing. All these transformations can be described with an affine transformation matrix.

$$T = [ \begin{matrix}a & b & X \\ c & d & Y \\ 0 & 0 & 1\end{matrix} ]$$

The muscle speed estimator calculates the Affine transform parameters between adjacent frames. There are several approaches to estimate these parameters. The optical flow solution is verified for good performance. The optical flow solution assumes that the brightness of moving pixels includes adjacent frames. We have [symbol] where U is the speed of the moving pixel in the image; [symbol] and [symbol] are the spatial gradients of intensity; and [symbol] is the temporal gradient. The spatial gradients of each pixel can be calculated by shifting and subtracting the image in the x and y directions by one pixel, respectively. The temporal gradient of each pixel is calculated by subtracting the intensity of the current frame from the last frame.

The pixel speed [symbol] , which is directly related to muscle speed, can be expressed as

$$U = [ \begin{matrix}a & b \\ c & d\end{matrix} ] ( \vec{x} - \vec{{x}_{0}} ) + [ \begin{matrix}X \\ Y\end{matrix} ] ,$$

where [symbol] is the coordinate of a pixel and a, b, c, d are rotational and shear parameters, and X and Y are translation parameters of the affine transformation. Having the [symbol] and [symbol] we can estimate U by calculating its least-square solution.

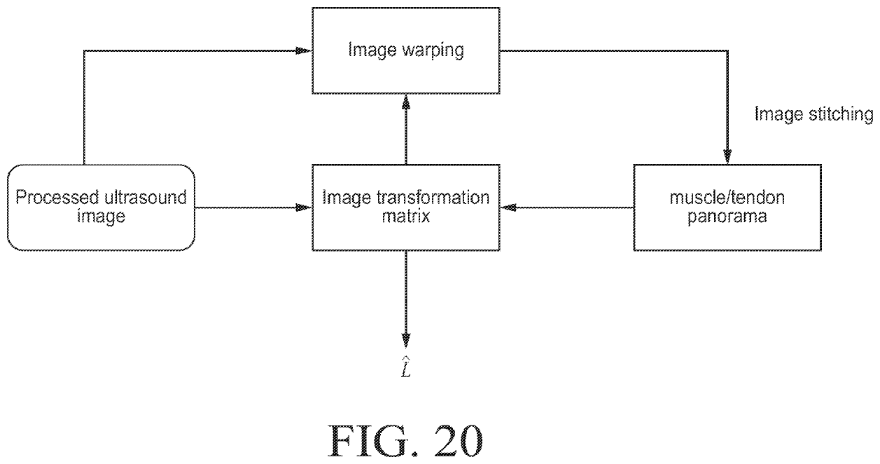

Muscle length estimator: The muscle length estimator performs two tasks simultaneously as shown in FIG. 20:
1. Building the muscle/tendon panorama; and
2. Finding the registration of the new ultrasound image frame.

The initial muscle/tendon panorama is the first stable ultrasound image of the target tissue. Upon receiving a new ultrasound image, the system will find its transformation from the existing panorama. Then the image warping module will warp the incoming image and stitch it to the existing panorama.

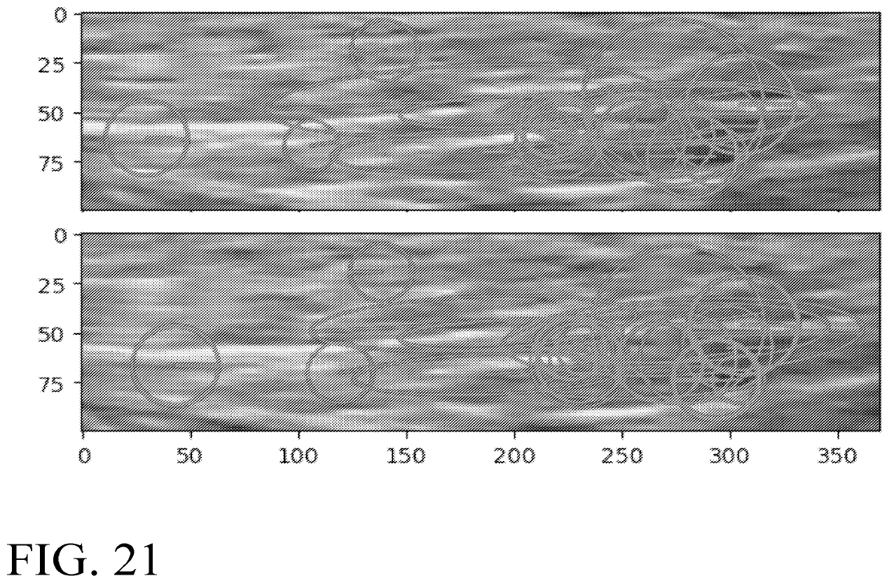

There are several ways to find the transformation. They include but are not exclusive:
1. Feature matching approach (an example is shown in FIG. 21); and
2. Deep learning-based transform estimator.

In the feature-matching approach, the pipeline finds the matching features from the panorama and the incoming frame first. Then, it estimates the transformation of the two images by the least square error estimation.

The deep-learning approach calculates the transform directly by, for example, spatial transformer neural network. In addition to a deep learning approach to estimate the affine transform parameters, a neural net that does not use affine flow features could be implemented. For example, a spatial CNN or U-NET architecture can extract high and low-level features to generate an estimate of the current muscle length state based on supervised learning. This could be done either through regression or classification-based methods. For example, for the classification method, the output of the model can be a probability distribution of the muscle length which is then input into the stochastic filter alongside the muscle velocity estimate from the affine flow transformation.

### Magnetomicrometry

In some example embodiments magnetomicrometry can be utilized to track tissue length and velocity changes in real time of the muscle or tendon for control (see FIG. 2). Like the marker-tracking process through ultrasound imaging, a pair of small coated magnetic beads are placed along the muscle belly or tendon. An array of magnetic field sensors are placed across the skin surface outside the body to track the changes in the magnetic field between the implanted magnets, relating to a relative distance between the bead pair.

In addition to tissue displacements and velocities, magnetic-based tracking can be used to measure tissue vibrations to indirectly measure force through shear wave and frequency measurements, as outlined earlier in the ultrasound section. In some embodiments, a magnetic marker is placed in a tendon. When loaded, the tendon's vibrations can be tracked through the magnetic field sensors measuring the vibrations and displacement of the bead. This vibration frequency [symbol] can be used to calculate muscle-tendon force [symbol] , using the equation F=(2fl)²*ρA, where A is the tendon cross-sectional area, l is the tendon length, and ρ is tendon density taken from the literature. Since tendon area, length, and density are fixed constants, a calibration exercise can be performed by measuring frequency and tendon force simultaneously for a static posture where force can be effectively estimated using an inverse dynamics calculation. Alternatively, as stated earlier in the ultrasound section, ultrasound or other imaging modalities can be employed to estimate tendon cross sectional area and length, and density set to a literature value.

In some embodiments, given an induced mechanical stimulus applied to the tendon to cause a shear wave, the wave can be measured using the disturbance in the beads from the stimulus. In some embodiments, more than one marker is placed either along or across the tendon, this enables the measurement of the wave speed difference between two or more markers. Tendon force [symbol] can then be estimated using the equation F=(c)²*ρA where A is the tendon cross-sectional area, and ρ is tendon density. These same examples can be applied to the muscle to estimate muscle activity. For the above, magnetic field image processing can employ synchronous demodulation tuned by a phase-lock loop to measure the frequency, amplitude, and phase.

### Wearable Acoustic Sensors

Beyond non-invasive ultrasound, there exists a vast array of other non-invasive sensing components that can serve as proxy to musculoskeletal dynamics. However, they typically have a more limited capacity to estimate relevant biophysical parameters compared to ultrasound and magnetomicrometry, requiring the use of machine learning techniques for their estimation, or the sensing component being employed in combination with other sensing modalities. The embodiments described herein leverage novel sensing mechanisms with unmatched sensitivity, either as standalone interfaces or coupled with local perturbation techniques to amplify the physiological effect for improved sensing resolution. These sensors are primarily made through thermal-drawing techniques. Preforms, consisting of the intended geometrical design and material organization of the fiber, are heated in a vacuum, until the preform naturally flows into a microscopic fiber. Pre-forms for these acoustic sensors would contain the piezoelectric component, such as poly(vinylidene fluoride-trifluoroethylene), that can be mixed with barium titanate ceramic particles. The pre-form also contains copper wires that are used as the electrical connection elements. Sandwiching the piezoelectric component are two sheets of polyethylene, and each component is encased in an elastomer for retaining flexibility.

### Measuring Natural Frequency of Tissue

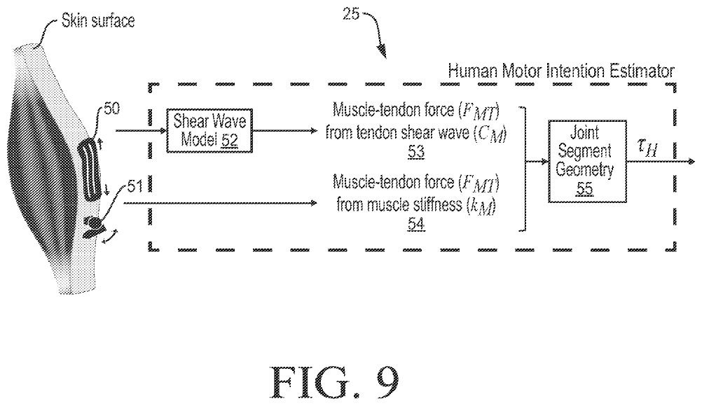

An acoustic sensor and an algorithm are used to estimate muscle force. The sensor can be placed across a tissue over the skin to estimate the force or load exerted under dynamic tasks by measuring the acoustic output (see FIG. 9). The sensor includes but is not limited to a sound transducer such as a microphone, a microphone fiber, or accelerometers. The algorithm analyzes either wave speed, vibrational frequency or a combination of both, from one or more sensors. In one sensor embodiment, microphone fibers are used to noninvasively track muscle-tendon acoustic frequency response. This is done through measuring natural acoustic properties of tendon and muscle when these organs are dynamically changing or during activation. The resulting acoustic behavior can estimate muscle activity or force output. In an additional embodiment, a sensor can be placed over the skin across a tendon or muscle with implanted markers such as the ones described earlier. The sensor tracks the wave speeds and vibration frequency of these beads to estimate tendon force. The beads may include but are not limited to titanium, stainless steel, hydrogel, ferromagnetic materials, or piezoelectric materials. In another embodiment, the measured acoustic behavior from the tissue can be combined with electromyography (EMG). The ratio between the amplitude of EMG and acoustic output is also a good indicator of muscle fatigue. Thus, combining with the other sensor signals in the system, the measured acoustics of the tissue can serve as inputs to a muscle-tendon state estimator supporting joint torque calculation and calibrations [16].

### Measuring Induced Shear Wave or Vibration

Another method to track force is to actuate tendon or muscle through vibration, using acoustic or ultrasound-based tapping, and to record frequency response through a microphone fiber that is placed across the skin surface. For example, in order to measure the force or load exerted by a superficial tendon under dynamic tasks, a sensor can be placed across the tendon over the skin, while a physical stimulus, such as a mechanical or acoustic tap, is delivered over the tendon. The physical stimulus generates a wave in the tendon that can be captured and analyzed by the sensor. This embodiment comprises a physical tapper, a sensor, and an algorithm. The tapper may include but is not limited to a piezoelectric micro-actuator, a vibrational actuator, a sound transducer such as a speaker, microphone fiber or ultrasound-based tapping. The sensor may include but is not limited to a sound transducer such as a microphone, a microphone fiber, ultrasound, or accelerometers. The algorithm analyzes either wave speed, vibrational frequency, or a combination of both, from one or more sensors. Additionally, or alternatively, a physical tapper, implanted beads in the tendon, a sensor, and an algorithm can be used to estimate tendon force. The sensor can be placed across a superficial tendon over the skin, while a physical stimulus is delivered over the tendon with implanted beads, such as the ones described earlier. The sensor tracks the wave speeds and vibration frequency of these beads to estimate tendon force. The beads may include, but are not limited to, titanium, stainless steel, hydrogel, ferromagnetic materials, or piezoelectric materials. The sensor may include, but is not limited to, an ultrasound patch, a sound transducer, a magnetic transducer, or a microphone fiber. The algorithm measures wave speed, vibrational frequency, or a combination of both, by using the implanted beads as tracking targets, or to aid in tendon dynamic measurements.

In another embodiment, the actuator can be used by itself, such that constant actuator palpations record a required force to reach a certain displacement across the tissue. The actuator can be integrated with a force and displacement sensor to receive these measurements. For example, this force and displacement relationship can estimate muscle stiffness in a local area. This estimation of muscle stiffness has a non-linear mapping to muscle-tendon force.

### Mechanomyography and Electromyography

In some example embodiments, a wearable sleeve with embedded sensors, for example in the form of fibers, can be used to non-invasively track muscle activation. The sensors include, but are not limited to, pressure sensors, strain sensors, EMG electrodes/sensors, or inertial measuring units (IMUs). The sensors can be either arranged in a high-density grid or localized where muscle activation is the highest. In one embodiment, sensors that sense their relative strain can be arranged in a grid across an area of high muscle palpation on the skin surface. As the muscle-tendon changes shape and the skin is stretched due to muscle belly activation, the change in strain across the surface can be correlated to muscle activity [17]. In another embodiment, force/pressure sensors can be oriented between the skin surface over a muscle and a sleeve interface, in order to detect the relative change in force, or pressure, due to changes in muscle stiffness to estimate muscle activity. Still further, pressure sensors can be arranged on the sole of the foot in a sock embodiment. This embodiment can track pressure in a high spatial resolution in the foot sole. The changes in pressure at the foot sole are used to estimate torque at the joint using an inverse dynamics calculation. Pressure sensors are placed at the positions where the human and the exoskeleton contact to measure the force between the exoskeleton and the user to assist the system calibration and control. Surface EMG electrode(s) on a wearable sleeve measures electrical activity of muscle as well [18]. A wearable sleeve with embedded multi-modal sensors listed above can provide a rich and thorough measurement of a muscle's electro-mechanical state.

### Joint-Based Prediction of Muscle Tendon Unit (MTU) State Information

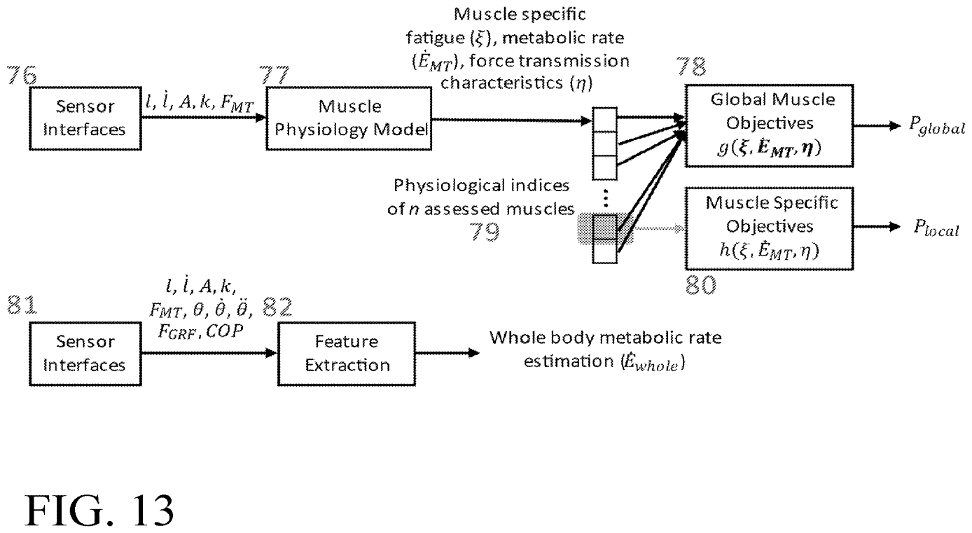

In some example embodiments, inertial measurement units (IMUs), comprised of magnetometers, accelerometers, and/or gyroscopes, may be used to determine the biological joint state—including, but not limited to, location, orientation, angular position, or angular velocity—of limb segments to which they are affixed (see FIG. 13). Some of these sensors may be integrated into the exoskeleton itself, while others may be placed on the user's limbs with elastic cuffs, adhesive, hook and loop connectors, or by integration into clothing. Using a musculoskeletal model calibrated to the wearer's specific anatomy, these IMUs can measure biological joint state in real-time. Other joint angle measurement techniques, such as goniometers and optical encoders, can also be used in lieu of IMUs. The exoskeleton's onboard joint-position sensors could also serve this purpose.

Such a calibrated musculoskeletal model with biological joint state sensory inputs could be used to estimate the total length of muscle-tendon units (MTUs) spanning the biological joint(s) for which joint state has been determined. When coupled with partial muscle-tendon information, such as muscle length or activation, derived from EMG, magnetomicrometry, ultrasound, mechanomyography, or other modalities listed earlier, these MTU length estimates can be used to estimate biological joint torques. By subtracting the total MTU length (from joint state measurements) from the measured muscle length (from muscle-tendon interfaces) in real time, tendon length changes can be estimated which when combined with tendon modulus information, can be used to estimate MTU force. Multiplying the MTU forces by each MTU's respective joint moment arm, biological joint torque can be estimated.

Biophysical muscle-tendon models typically comprise a series-elastic element to the muscle CE, representing tendon elasticity. Measuring joint position and velocity and estimating the total muscle-tendon lengths and velocities does not determine the individual lengths and velocities of each muscle and tendon individually. Thus, estimating the total length of a MTU via joint position measurements only approximates total muscle length with the assumption that there exist no series-elastic element within the MTU. Only when using this simplification can one deterministically estimate muscle length and velocity based on joint state measurement. Using a biophysical muscle model (e.g., Hill model), MTU force can then be estimated with the muscle length and speed derived from these joint state measurements, and muscle activation derived from the muscle-tendon interface measurements (EMG, magnetomicrometry, ultrasound, mechanomyography, or other modalities listed). Once again, by multiplying the MTU forces by each MTU's respective joint moment arm, biological joint torque can be estimated.

### Human Motor Intention Estimator

This section describes the module termed Human Motor Intention Estimator shown in FIG. 1. The Human Motor Intention Estimator module receives information from at least one muscle-tendon interface, and potentially other wearable sensors, and estimates muscle-tendon joint torque, muscle CE mechanical power, muscle metabolic power, or muscle efficiency. In some embodiments, at least one of these real-time estimates of at least one muscle-tendon unit are then inputted into an Augmentation Strategy module that computes in real time at least one augmentation joint torque. In some embodiments, at least one of these real-time estimates of at least one muscle-tendon unit are then inputted into an Augmentation Strategy module that computationally adapts across multiple motor cycles towards at least one optimal torque profile. The Augmentation Strategy module sends such torque commands to the wearable device's torque controller. A control framework is presented herein wherein a powered exoskeleton applies closed-loop (servo) control of one or more augmentation torques under computer control about an exoskeletal joint or joints. In some example embodiments, the time between at least one muscle-tendon interface measuring at least one physiological signal and applying the at least one augmentation joint torque to the at least one actuated joint of the wearable robot is less than 100 ms. In some example embodiments, the time between at least one muscle-tendon interface measuring at least one physiological signal and applying the at least one augmentation joint torque to the at least one actuated joint of the wearable robot is less than or equal to 100 ms.

Examples are provided herein of biological neuromechanical structures comprised of muscles, tendons, and their corresponding joints. Many of the implementations described herein refer to the ankle joint, as it is an important joint for locomotion in humans, and a particular one that benefits from wearable devices such as exoskeletons. However, it should be understood that this application is not limited to any particular joint, but is general to any biological joint and wearable robot.

To get an estimation of human motor intention, in some embodiments we describe different methods to estimate biological human torque based on biophysical signals measured using muscle-tendon sensory interfaces and potentially other wearable sensors. In order to estimate biological torque about a joint, the force from muscle-tendons spanning the joint are required. As muscles and tendons are in series, only one force measurement is required, either borne by the muscle or borne by the tendon. For simplicity, this force value, as measured either from the muscle or tendon, will be termed the muscle-tendon force. To estimate the torque contribution about a biological joint resulting from a muscle-tendon force, a model of the segmental morphologies and dynamics can be used to estimate the moment arm about which the muscle-tendon acts about the joint. The segmental dynamics can be estimated based on measurements from on-board sensors like IMUs. Torque can be estimated using an a priori knowledge of joint morphologies and the resulting moment-arm relationships with measured joint angle.

In some example embodiments, to get an estimation of the muscle-tendon force requires the measurement of the length and velocity of the muscle, as well as the muscle's activation. In some example embodiments, using a biophysical muscle model, these three inputs are then used to compute muscle-tendon force in real time. Methods are described herein to obtain these three measurements to estimate muscle force within the Human Motor Intention Estimator module.

Example embodiments include marker-based and non-invasive approaches. Within the marker-based approach, muscle-tendon force can be obtained using implanted:
1) Ultrasound markers to estimate muscle length and velocity though fascicle tracking, as well as muscle activation through a muscle vibration measurement
2) Magnetic markers to estimate muscle length and velocity though fascicle tracking, as well as muscle activation through a muscle vibration measurement
3) Ultrasound markers to estimate muscle length and velocity though fascicle tracking, and EMG to estimate muscle activation
4) Magnetic markers to estimate muscle length and velocity though fascicle tracking, and EMG to estimate muscle activation
5) Ultrasound markers to estimate muscle length and velocity though fascicle tracking, and strain myography to estimate muscle activation
6) Magnetic markers to estimate muscle length and velocity though fascicle tracking, and strain myography to estimate muscle activation
7) Ultrasound markers to estimate muscle length and velocity though fascicle tracking, and acoustic myography to estimate muscle activation
8) Magnetic markers to estimate muscle length and velocity though fascicle tracking, and acoustic myography to estimate muscle activation

Within the non-invasive approach, muscle force can be obtained using:
1) Non-invasive ultrasound to estimate muscle length and velocity though fascicle tracking, and non-invasive ultrasound to estimate muscle activation
2) Non-invasive ultrasound to estimate muscle length and velocity though fascicle tracking, and EMG to estimate muscle activation
3) Non-invasive ultrasound to estimate muscle length and velocity though fascicle tracking, and strain myography to estimate muscle activation
4) Non-invasive ultrasound to estimate muscle length and velocity though fascicle tracking, and acoustic myography to estimate muscle activation
5) Mechanomyography palpation to measure muscle stiffness to estimate muscle force

In addition, a real-time method that measures muscle length in combination with a sensor that detects joint angle, or angles, can be used to directly estimate muscle force. For a monoarticular muscle, the angle of the joint across which the muscle-tendon spans is translated to a muscle-tendon unit (MTU) total length using morphological data related to the muscle-tendon's origin and insertion locations relative to the joint axis. A similar method can be used for muscle-tendons that span more than a single joint, except all the joint angles are required to estimate the MTU's total length. In addition to the MTU total length estimate in real time, the muscle length is estimated using an ultrasound measurement. The length of the tendon is then equal to the MTU length minus the muscle length. Given the resting tendon slack length, the tendon strain is estimated, which in turn is used to predict tendon force using a nonlinear tendon equation relating tendon force to tendon strain. As the muscle and tendon are in series, the force is the same across them, thereby giving the respective muscle force with these two parameters as inputs.

In another embodiment, a muscle-tendon force estimate is derived from a direct measurement of tendon length. Specifically, tendon length is measured in real time, and given the resting tendon slack length, the tendon strain is estimated, which in turn is used to predict tendon force using a nonlinear tendon equation relating tendon force to tendon strain. Alternatively, tendon stiffness can be measured in real time, an employed to calculate tendon force. Given the nonlinear relationship between tendon stiffness and tendon force, by knowing the stiffness one can estimate force.

With these methods, tendon force can be obtained using:
1) Implanted ultrasound markers for real-time tendon length measurements
2) Implanted magnetic markers for real-time tendon length measurements
3) Non-invasive ultrasound for real-time tendon length measurement
4) Non-invasive ultrasound tissue elastography for tendon stiffness estimation

Another method to estimate muscle-tendon force, as measured by the force produced by a tendon, is from measurements of tendon vibration frequency to estimate tendon force, using the equation F=(2fl)²*ρA, where A is the tendon cross-sectional area, l is the tendon length, and ρ is tendon density. Tendon stress, indicative of loading or force, increases as a function of tendon frequency and length.

With this method, tendon force can be obtained using:
1) Implanted ultrasound markers to estimate tendon vibration frequency
2) Implanted magnetic markers to estimate tendon vibration frequency
3) Ultrasound to estimate tendon vibration

Another method to estimate muscle-tendon force, as measured by the force produced by a tendon, is from shear-wave measurements generated by a palpation actuator mechanism. The palpation actuator generates a shear-wave that is measured as a speed c, converted to tendon force, or muscle-tendon force, using the equation F=(c)²*ρA, where A is the tendon cross-sectional area, and ρ is tendon density.

Within this method, tendon force can be obtained using:

1) Implanted ultrasound markers and actuator palpation to estimate tendon wave speed
2) Implanted magnetic markers and actuator palpation to estimate tendon wave speed
3) Acoustic sensing and actuator palpation to estimate tendon wave speed
4) Strain sensing and actuator palpation to estimate tendon wave speed

### Inverse Dynamics

Another method to estimate human biological torque is from inverse dynamics. Joint torque around a specific joint can be measured indirectly through the application of inverse dynamics, where inertial properties of body segments, combined with their configuration and accelerations, are used to solve the force-moment balance equation. Additionally, this method requires knowledge of interaction forces between the body being analyzed and the environment. For applications in gait, this is sometimes obtained through six-axis load cells which measure the reaction forces between the ground and subject. With knowledge of the location, magnitude, and direction of these reaction forces, and additionally inertial properties of body segments, their configuration and accelerations, joint torques can be computed. Using this knowledge, and knowledge of the torque being applied by the exoskeleton, the biological human torque being applied by the wearer can be determined by simple subtraction. With sufficiently small delays between exoskeleton torque measurement and total joint torque estimation, a close approximation of the wearer's instantaneously commanded biological torque can be obtained.

These ground reaction forces can be measured explicitly, using an instrumented shoe with multi-axis sensing. In another embodiment, these forces could be estimated using a pressure-sensitive insole, and machine-learning algorithms that estimate ground reaction shear forces based on the time-history of the center of pressure and the magnitude of the orthogonal ground reaction force.

In some example embodiments, a pressure-sensitive insert or liner between the body and the exoskeleton, combined with body joint sensors, can be used to estimate human joint torque using an inverse dynamics calculation. In one example, the vertical component of the ground reaction force vector could be estimated using a pressure-sensitive insole positioned between the biological foot and an exoskeleton-actuated midsole of a motorized-ankle shoe. A machine-learning algorithm could then be used to estimate ground reaction shear forces based on the time-history of the center-of-pressure and the vertical component (orthogonal) of the ground reaction force vector. Combining the estimated ground reaction force vector with its center-of-pressure point of application, and the perpendicular distance from the force vector's line of action and the ankle axis of rotation (moment arm), ankle biological human torque can be estimated. The moment arm can be obtained through shoe- and exo-mounted IMU sensors that measure joint angles, as well as joint morphologies that determine the joint angle-moment arm relationship. Thus, the orthogonal pressure and its distribution, or center-of-pressure, relative to a known axis of rotation (obtained through ankle position sensing) could be used to compute how strongly the wearer is pushing through the exoskeleton into the ground, or a biological human torque estimate. This torque sensing modality could be applied to any exoskeletal application where the robot lies between the wearer and the object the wearer is interfacing with. The human torque could be used as a control target which can then be used as an input to a power-steering exoskeleton controller wherein the applied augmentation torque is correlated to the biological human joint torque. The details of this type of controller are described in detail within the subsequent section.

### Controllers: Biological Torque Control.

In some example embodiments, after estimating muscle-tendon force, the Human Motor Intention Estimator (FIG. 1, element 25) computes the biological human torque. The muscle-tendon force, multiplied by the biological joint muscle-tendon moment arm, is used to compute intended biological human torque about a joint. The muscle-tendon moment arm about any biological joint can be estimated by measuring its joint angle and inputting that measured angle into an accurate anatomical model of the biological limb to effectively estimate the moment arm at that measured angle.

An example using marker tracking with ultrasound or magnetomicrometry to estimate muscle-tendon force is illustrated in FIG. 2. FIG. 2 shows two markers within or on a muscle, and two markers within or on a tendon for the estimation of muscle strain and tendon strain, respectively. As described in connection with FIG. 2, in some example embodiments, this marker tracking methodology using ultrasound or magnetomicrometry is employed to estimate muscle-tendon force. In a first method, marker tracking is used to measure muscle length and speed. A biophysical muscle model is then used to estimate muscle-tendon force (FIG. 2, elements 32, 34, 36, 37) using, additionally, a measurement of muscle activation. In a second method, tendon bead tracking is used to estimate tendon strain. With an a priori knowledge of tendon material modulus, muscle-tendon force can be estimated.

Another example of a muscle-tendon interface is shown in FIG. 3. In this approach muscle is imaged using non-invasive ultrasound without requiring the use of implanted beads. Here feature extraction with ultrasound is used to estimate muscle fascicle movements.

The muscle-tendon interfaces shown in FIG. 2 and FIG. 3 are just representative examples. As indicated in this Human Motor Intention Estimator section, the input parameters of the muscle-tendon model can come from any combination of the muscle-tendon interfaces described.

In some example embodiments, the Human Motor Intention Estimator described in FIG. 1 receives information from at least one muscle-tendon interface, and potentially other wearable sensors, to estimate muscle CE mechanical power. CE power is computed as the product of muscle-tendon force and CE velocity. As described earlier, muscle CE velocity can be estimated using a plurality of implanted ultrasound or magnet markers placed on or within an individual muscle, and then computing the time rate of change of the relative distance between the implanted markers. In addition, muscle CE velocity can be estimated non-invasively using ultrasound doppler velocimetry as described earlier. The CE muscle power of a muscle or muscles are then sent to the Augmentation Strategy module 26 shown in FIG. 1 where exoskeleton torque and power targets are adjusted so as to minimize muscle CE power.

In another embodiment, the Human Motor Intention Estimator estimates muscle metabolic power. By measuring the length, speed, and activation of muscles through muscle-tendon interfaces and intrinsic sensors described earlier, it is possible to accurately estimate the metabolic energy expenditure from specific muscles. Multiple muscle energy expenditure estimations can lead to an accurate model of whole-leg metabolism as well. Utilizing a muscle energetics model, the metabolic cost of muscle dynamics can be inferred. The controller applies the estimated muscle energetics to calculate the torque or power needed to reduce the metabolic energy expended by a muscle or muscles. This method provides a faster metabolic closed feedback loop compared to conventional methods such as a respirometry or indirect calorimetry system, which require continuous recording on a minute-scale.

Due to the high temporal resolution of the aforementioned sensors and methods for predicting force, length, speed and activation of a muscle, the metabolic energy of a muscle can be computed quickly such that a response from the exoskeleton actuator can occur in real-time. For example, the total energy expenditure of the muscle can be calculated through the following equation:

$$\dot{E} = \dot{{h}_{AM}} + \dot{{h}_{SL}} + \dot{{w}_{CE}} \qquad ( 1 )$$

Here, [symbol] represents the activation and maintenance heat rates of the muscle, the [symbol] is the shorten or lengthen heat rate, and [symbol] is the mechanical work rate of the muscle. These variables depend on previously defined parameters for specific muscles, such as the distribution of fast-twitch and slow-twitch fibers, and, for example, Hill muscle model constants. However, some of these values change based on the dynamics of the muscle in real-time. The [symbol] is dependent on the fiber distribution (% FT) of the muscle being targeted:

$$\dot{{h}_{AM}} = 1.28 * ( \% FT ) + 25 \qquad ( 2 )$$

The [symbol] relies on the model coefficients and the muscle fiber velocity which determine the shortening and lengthening heat rates:

$$Shortening \text{:} \dot{{h}_{SL}} = - {α}_{S ( ST )} * ( {\tilde{V}}_{CE} ) * ( 1 - ( \frac{\% FT}{100} ) ) - {α}_{S ( FT )} * ( {\tilde{V}}_{CE} ) * ( \frac{\% FT}{100} ) \qquad ( 3 )$$

$$Lengthening \text{:} \dot{{h}_{SL}} = {α}_{L} * ( {\tilde{V}}_{CE} ) \qquad ( 4 )$$

The [symbol] is the normalized muscle fiber velocity based on the maximum fiber velocity and [symbol] represents the Hill muscle-model coefficients. The final term [symbol] can be calculated through the following equation:

$$\dot{{w}_{CE}} = - \frac{{F}_{CE} {V}_{CE}}{m} \qquad ( 5 )$$

Here, m represents the mass of the muscle, [symbol] is the muscle-tendon force, and [symbol] is the contractile velocity.

An example of utilizing the above equations in combination with the muscle-tendon interfaces is through the ultrasound interface. The ultrasound interface can measure muscle fiber length, velocity, and force using the methods outlined earlier. In these embodiments, the Human Motor Intention Estimator 25 then uses this real-time estimate of muscle-tendon state, as well as muscle model coefficients, to estimate the total energy expenditure, or rate of metabolic energy expenditure, of one or multiple muscles. The metabolic energetics of the muscle or muscles are then sent to the Augmentation Strategy module 26 where torque and power targets are adjusted so as to minimize muscle metabolic power.

In another embodiment, the Human Motor Intention Estimator 25 estimates muscle efficiency, computed as the ratio of CE muscle power divided by metabolic power using the estimation methods defined earlier. The efficiency of a muscle or muscles is then sent to the Augmentation Strategy module 26 where torque and power exoskeleton targets are adjusted so as to maximize muscle efficiency. Alternatively, a priori knowledge of the functional relationship between muscle efficiency and normalized muscle shortening velocity (normalized by the maximum shortening velocity) can be used to estimate efficiency from a real time measurement of a muscle's shortening velocity determined using, for example, ultrasound doppler velocimetry described earlier.

### Controllers

### Power Steering Control: Biological Joint Torque Control

From the muscle-tendon interfaces 23 and intrinsic sensors 24 described earlier, a number of physiological parameters from the wearer can be obtained, such as neural activation, muscle length, muscle velocity, muscle-tendon force, among others. A biophysical model is used to relate the signals measured from the interfaces to the physiological parameters. For example, for the acoustic myography sensor, a shear wave physical model relates sound waves to the physiological parameter of muscle-tendon force. These parameter(s) serve as input(s) to a Human Motor Intention Estimator 25 that computes a torque signal representing the applied biological torque from the human. Intrinsic parameters from intrinsic sensors 24 at the exoskeleton device, such as ground reaction force, center-of-pressure, and joint angles, can also serve as inputs to the Human Motor Intention Estimator 25. The human biological torque signal computed by the Human Motor Intention Estimator 25 is later modified through a constant or adaptive gain function by the Augmentation Strategy Module 26 that computes the augmentation torque command to be sent to the Torque Controller 27 of the exoskeleton device. The Augmentation Strategy Module 26 computes the level of assistance, or augmentation torque, according to the application, for example, if it's used to assist someone with a neurological condition or if it's used for augmentation purposes. The Augmentation Strategy Module 26 relates the biological torque input to the augmentation torque output using linear or nonlinear functional relationships. In some example embodiments of biological muscle torque control, augmentation torque command for an exoskeleton joint is functionally related to the human biological torque about the parallel biological joint such that augmentation torque monotonically increases/decreases as the human biological torque increases/decreases in real time, respectively.

The Augmentation Strategy Module 26 could also compute augmentation torque from the human biological joint torque input based upon phase of gait, gait speed or underlying terrain variations (stairs, incline, rocky surface, etc.). For example, the gain that amplifies the human torque to compute the augmentation torque could be increased by the augmentation-strategy control block with increasing gait speed, or for a ground surface that steadily increases in steepness, or when the exoskeletal user transitions from a level walking surface to stairs. The exoskeletal control system would detect these changes in gait phase, gait speed and underlying terrain from sensory information obtained from the muscle-tendon neural interfaces 23 and/or from intrinsic sensors 24.

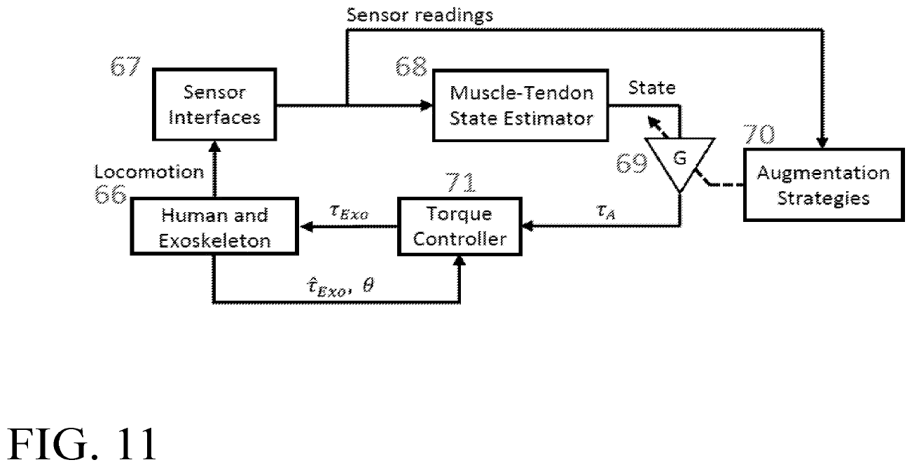

The augmentation torque commands computed by the Augmentation Strategy Module 26 are input into the exoskeleton Torque Controller 27 as control targets, where the motor driver sends a current command to each respective exoskeleton actuator. This Torque Controller 27 can run a feedback loop to servo to the augmentation torque command for each exoskeleton joint. The control diagram for this control strategy is described in FIG. 11.

Similar to the power steering in a car, which measures the torque applied through the steering column by the driver and applies a proportional torque in parallel to the driver's mechanical input, the biological human joint torque can be used to determine how much additional augmentation torque the exoskeleton should provide to amplify human motor capability.

The control diagram of FIG. 11 focuses on augmentation-based objectives for exoskeleton control using the power steering framework. First, the dynamics of the human and current state of the exoskeleton 66 are estimated through the multiple Sensor Interfaces 67, the totality of the Muscle-Tendon Neural Interfaces and Intrinsic Sensors described earlier in FIGS. 2 through 10. Sensory information from the Sensor Interfaces is then input into the Muscle-Tendon State Estimator 68, a part of the Human Motor Intention Estimator 25 from FIG. 1, to generate an estimation of the torque output from the human about a joint or joints. The human torque goes into an adaptive or constant functional relationship 69 which is performed by the Augmentation Strategies 70 computational module to produce an augmentation torque target. Augmentation Strategies include but are not limited to minimization of muscle fatigue, or amplifying muscle force, for example, to augment jumping height. Readings from the Sensor Interfaces are used by the Muscle-Tendon State Estimator to estimate biological muscle torque in real time. With biological torque as in input, the Augmentation Strategies then compute augmentation torque, or torques. The augmentation torque target is then input into the Torque Controller, which applies the torque to the human joint through a closed-loop wearable robotic control 71. On-board sensors of the wearable robot feedback the joint angle and output robotic torque back into the controller to close the loop on the augmentation torque command target 71. Without loss of generality, the augmentation torque determined by the Augmentation Strategy may be derived from a time-varying, joint equilibrium and impedance.

### Power Steering Control: Muscle CE Power Control

For the muscle CE power control, the biological muscle CE power and its corresponding joint power can be derived from each muscle and joint to contribute toward a commanded exoskeletal power. An example of a muscle-to-joint derivation is shown below in Equations 6-12.

The mechanical power contributed by the nth MTU spanning joint i is equal to:

$${P}_{n , i}^{MTU} = ( {T}_{n , i}^{MTU} ) ( {\dot{θ}}_{i} ) \qquad ( 6 )$$

where

$${T}_{n , i}^{MTU}$$

is the torque about joint i from the nth MTU, and [symbol] is the angular velocity of joint i.

The CE mechanical power from the nth muscle-tendon unit spanning joint i is equal to:

$${P}_{n , i}^{CE} = ( {P}_{n , i}^{MTU} ) ( \frac{( {F}_{n} ) {V}_{n}^{CE}}{( {F}_{n} ) {V}_{n}^{MTU}} ) \qquad ( 7 )$$

where

$${F}_{n}^{} , {V}_{n}^{CE} and {V}_{n}^{MTU}$$

are the muscle force, CE velocity, and MTU velocity of the nth muscle, respectively.

Combining equations (6) and (7), and simplifying we have:

$${P}_{n , i}^{CE} = ( ( {T}_{n , i}^{MTU} ) ( {\dot{θ}}_{i} ) ) ( \frac{{V}_{n}^{CE}}{{V}_{n}^{MTU}} ) \qquad ( 8 )$$

The CE mechanical power from all muscle-tendon units spanning joint i is then equal to:

$${P}_{i}^{CE} = {\sum }_{n = 1}^{\# Muscles} [ ( ( {T}_{n , i}^{MTU} ) ( {\dot{θ}}_{i} ) ) ( \frac{{V}_{n}^{CE}}{{V}_{n}^{MTU}} ) ] \qquad ( 9 )$$

After the Human Motor Intention Estimator 25, the specific power information is fed into the Augmentation Strategy as an exoskeleton in parallel to joint i applies an exoskeletal power about joint i that is functionally related to

$${P}_{i}^{CE} ,$$

linearly or nonlinearly, or

$${P}_{i}^{EXO} is a function of ( {\sum }_{n = 1}^{\# Muscles} [ ( ( {T}_{n , i}^{M T U} ) ( {\dot{θ}}_{i} ) ) ( \frac{{V}_{n}^{C E}}{{V}_{n}^{M T U}} ) ] ) \vert {V}_{n}^{M T U} \vert > 0 \qquad ( 10 )$$

By convention, positive velocity refers to a muscle contraction (shortening) velocity.

As a simple example, consider a unidirectional ankle exoskeleton that actuates during ankle controlled dorsiflexion and powered plantar flexion from mid to late stance. For this example, the calf muscle comprising soleus and gastrocnemius aspects span the biological ankle joint. Assuming a simple linearly proportional relationship between exoskeleton mechanical power and CE muscle power, we have:

$${P}_{a n k l e}^{EXO} = ( {T}_{a n k ι e}^{EXO} ) ( {\dot{θ}}_{a n k l e} ) = K ( {Σ}_{n = 1}^{2} [ ( ( {T}_{a n k l e}^{m uscle n} ) ( {\dot{θ}}_{a n k i e} ) ) ( \frac{{V}_{n}^{CE}}{{V}_{n}^{M T U}} ) ] ) \qquad ( 11 )$$

where K is a proportionality constant set equal to one for this simple case. As noted in equation 10, the exoskeleton only applies torque when

$$\vert {V}_{n}^{M T U} \vert > 0 ,$$

or when the calf MIU is actively applying either negative or positive mechanical power about the ankle joint.

Simplifying,

$${T}_{a n k ι e}^{EXO} = K ( ( ( {T}_{a n k l e}^{s o l e u s} ) ) ( \frac{{V}_{s o l e u s}^{CE}}{{V}_{soleus}^{MTU}} ) + ( ( {T}_{a n k l e}^{g a s t r o c} ) ) ( \frac{{V}_{g a s t r o c}^{CE}}{{V}_{g a s t r o c}^{MTU}} ) ) \qquad ( 12 )$$

When the soleus and gastrocnemius generate force isometrically,

$${V}_{soleus}^{CE} = {V}_{gastroc}^{CE} = 0 , and {T}_{ankle}^{EXO} = 0.$$

Since a muscle can generate force isometrically at a very low metabolic rate, the exoskeleton applies zero augmentation torque.

In distinction, when

$${V}_{soleus}^{CE} = {V}_{soleus}^{MTU} , and {V}_{gastroc}^{CE} = {V}_{gastroc}^{MTU} ,$$

the calf muscle fibers contribute all the mechanical power, with the Achilles tendon contributing zero power. For this case.

$${T}_{a n k ι e}^{EXO} = {T}^{s o l e u s} + {T}^{g a s t r o c}$$

which is equal to the total biological torque from the calf muscle about the ankle joint. Since metabolic costs are high when a muscle contracts and produces positive power, the exoskeleton applies 100% positive torque to completely eliminate CE muscle positive power. During a controlled dorsiflexion motor phase when the calf MTU applies negative power, the exoskeleton applies 100% negative torque when the muscle absorbs all the mechanical energy and the Achilles tendon absorbs little to no power. Such a braking exoskeletal control would mitigate injury and metabolic cost.

Intermediate values of

$${T}_{a n k ι e}^{EXO}$$

are applied when both the soleus and gastrocnemius muscle CE's, as well as the Achilles tendon, contribute either positive or negative power about the ankle joint.

Since in the controlled dorsiflexion phase of walking, and the early phase of ankle powered plantar flexion, the calf muscle generates largely isometric force, followed by a period of rapid CE shortening during the late phase of powered plantar flexion, the peak of the exoskeletal applied plantar flexion torque would be delayed from the peak of the biological calf muscle ankle torque. For an ankle exoskeleton, the delay in peak torque maximally reduces walking metabolic energy while also keeping exoskeletal torque and actuator mass low.

In a simplified embodiment,

$${T}_{a n k ι e}^{EXO}$$

would only be applied during power plantar flexion in walking and running, when the calf muscle MTU's apply positive power, since positive CE contractions are far more expensive metabolically than negative CE contractions that occur during controlled dorsiflexion. Such a simplification would further reduce the size and weight of the wearable ankle-foot exoskeleton. In another simplification,

$${T}_{a n k ι e}^{EXO}$$

could be applied that only includes soleus CE power contributions, or gastrocnemius contributions, but not both.

For exoskeletal control embodiment described by equation 10, the neuromuscular interface design must estimate muscle force, CE velocity, MTU velocity, and muscle moment arm for one or more muscles spanning each actuated joint. Muscle-tendon interfaces designed to estimate these variables are described in the earlier section Human Motor Intention Estimator.

The control diagram of FIG. 11 focuses on augmentation-based objectives for exoskeleton control using the power steering framework where muscle CE power serves as an input. First, the dynamics of the human and current state of the exoskeleton 66 are estimated through the multiple Sensor Interfaces 67, the totality of the Muscle-Tendon Neural Interfaces and Intrinsic Sensors described earlier in FIGS. 2 through 10. Sensory information from the Sensor Interfaces is then input into the Muscle-Tendon State Estimator 68, a part of the Human Motor Intention Estimator from FIG. 1, to generate an estimation of the muscle CE power about a joint or joints. The muscle CE power goes into an adaptive or constant functional relationship 69 which is performed by the Augmentation Strategies 70 computational module to produce an augmentation power or torque target as described, for example, by equation 10. Augmentation Strategies include but are not limited to minimization of muscle CE power or work, for example, to reduce metabolic cost. Readings from the Sensor Interfaces are used by the Muscle-Tendon State Estimator to estimate biological muscle CE power in real time. With biological CE power as in input, the Augmentation Strategies then compute augmentation torque, or torques. The augmentation torque target is then input into the Torque Controller, which applies the torque to the human joint through a closed-loop wearable robotic control 71. On-board sensors of the wearable robot feedback the joint angle and output robotic torque back into the controller to close the loop on the augmentation torque command target 71. Without loss of generality, the augmentation torque determined by the Augmentation Strategy may be derived from a time-varying, joint equilibrium and impedance.

### Power Steering Control: Joint Power Control

To maximize exoskeleton accessibility, a controller that only uses information from non-invasive intrinsic sensors such as IMUs 24 is preferred. Therefore, a simplified embodiment utilizes kinematic features of legged motion to approximate mechanical joint power only using information from on-board intrinsic sensors. The following describes an exoskeleton joint controller that relates exoskeletal augmentation torque with metabolically expensive propulsive mechanics.

The mechanical power spanning joint i is equal to:

$${P}_{i} = ( {T}_{i} ) ( {\dot{θ}}_{i} ) \qquad ( 13 )$$

where [symbol] is the net torque about joint i, and [symbol] is the angular velocity of joint i.

As described in an earlier section Human Motor Intention Estimator, [symbol] can be accurately estimated using non-invasive sensor interfaces. Metabolic costs are high when a muscle produces positive power and muscle dynamics approximately correspond to when the joint produces positive power. Therefore, the exoskeleton joint torque is controlled as:

$${T}_{i}^{EXO} = G ( H ( {P}_{i} , ϵ ) , γ ) \qquad ( 14 )$$

where [symbol] is a step function with a threshold [symbol] and [symbol] is a simple proportionality constant (gain) or a non-linear function with additional control inputs [symbol] allows the exoskeleton to produce assistive torque only when [symbol] is positive (or beyond [symbol] ). This ensures exoskeleton assistance is only delivered when a joint produces positive work. Furthermore, this allows a user to naturally perform negative work cycles that are critical for efficient legged motions. Additional control inputs [symbol] such as the joint kinematics [symbol] and [symbol] can be employed to provide biomimetic assistance while accounting for torque characteristics of biological joints.

Estimating accurate [symbol] can be achieved through inverse kinematics and dynamics which, for an ankle-foot exoskeleton, requires previously described calibrated, insole sensor inserts. An approach that does not require inverse dynamics to estimate the propulsive mechanisms of an ankle joint is described herein. Ankle joint net torque can be approximated as:

$${T}_{ankle} = I ( φ , μ ) {\ddot{θ}}_{ankle} \qquad ( 15 )$$

where [symbol] is a mechanical inertia varied by overall joint leg kinematics [symbol] and external factors [symbol] such as gait phase. Combining and simplifying equations 13 and 15, we have:

$${P}_{ankle} = I ( φ , μ ) {\ddot{θ}}_{ankle} {\dot{θ}}_{a n k l e} \qquad ( 16 )$$

From equation 16, [symbol] ankle provides an approximation of mechanical power directionality (negative or positive) and relative amplitude. For an ankle exoskeleton, this propulsive mechanic feature encodes control inputs for exoskeletal augmentation torque. For example, substituting [symbol] in equation 14 with the specific propulsive mechanic feature [symbol] we have:

$${T}_{i}^{EXO} = G ( H ( σ , ϵ ) , γ ) \qquad ( 17 )$$

Equation 17 provides a general control design architecture similar to equation 9. For example, threshold [symbol] can be set so that an exoskeleton provides assistance for propulsive mechanics [symbol] while remaining passive during compliant dynamics [symbol] or sway [symbol] . As an additional control input [symbol] for example, environment independent gait information such as single and double leg support phases can be employed to account for [symbol] variance. Also, the joint kinematics [symbol] and [symbol] can be employed to provide biomimetic assistance while accounting for joint kinematics-dependent torque characteristics of biological joints. Note that such an exoskeletal controller that uses a propulsive mechanic feature provides universal assistance to any legged motions that involve a propulsion phase (propulsive positive work that is metabolically expensive) including walking at different inclinations and speeds, running, stair ascent, and jumping. The minimum sensory requirements for this controller are joint kinematics which can be easily obtained from on-board sensors such as joint encoders or IMUs.

Depending on the information generated from muscle-tendon interfaces described in the Human Motor Intention Estimator, the joint power control for an exoskeleton can be realized in multiple embodiments ranging from directly using net mechanical power values described by equation 14, to using an approximation of propulsive mechanical joint power as in equations 16 and 17. The joint or muscle kinematics using both joint angle velocity and acceleration, as well as muscle fiber velocity and acceleration can be used to estimate power and torque of either the joint or muscle, respectively.

The control diagram of FIG. 11 focuses on augmentation-based objectives for exoskeleton control using the power steering framework where biological joint power serves as an input. First, the dynamics of the human and current state of the exoskeleton 66 are estimated through the multiple Sensor Interfaces 67. Sensory information from the Sensor Interfaces is then input into the Muscle-Tendon State Estimator 68, a part of the Human Motor Intention Estimator 25 from FIG. 1, to generate an estimation of the biological joint power. The joint power goes into an adaptive or constant functional relationship 69 which is performed by the Augmentation Strategies 70 computational module to produce an augmentation torque target as described, for example, by equation 17. Augmentation Strategies include but are not limited to minimization of biological joint power or work, for example, to reduce metabolic cost. Readings from the Sensor Interfaces are used by the Muscle-Tendon State Estimator to estimate biological joint power in real time. With biological joint power as in input, the Augmentation Strategies then compute augmentation torque, or torques. The augmentation torque target is then input into the Torque Controller 27, which applies the torque to the human joint through a closed-loop wearable robotic control 71. On-board sensors of the wearable robot feedback the joint angle and output robotic torque back into the controller to close the loop on the augmentation torque command target 71. Without loss of generality, the augmentation torque determined by the Augmentation Strategy may be derived from a time-varying, joint equilibrium and impedance.

### Power Steering Control: Metabolic Cost Controller

By measuring the length, speed, force and activation of muscles through muscle-tendon interfaces 23 and intrinsic sensors 24 described earlier, it is possible to estimate the metabolic energy expenditure from specific muscles. Multiple muscle energy expenditure estimations can lead to an accurate model of whole-leg metabolism as well. Utilizing a muscle energetics model, the metabolic cost of muscle force generation for dynamic activities can be estimated in real time. Using the real time muscle metabolic consumption of a muscle or a set of muscles, the Augmentation Strategy Module 26 calculates the exoskeletal augmentation torque or power needed to reduce the metabolic energy expended by the exoskeletal wearer. This method provides a faster metabolic closed feedback loop compared to conventional methods such as a respirometry or indirect calorimetry system, which require continuous recording on a minute-scale.

Due to the high temporal resolution of the aforementioned sensors and methods for predicting force, length, speed and activation of a muscle, the metabolic energy of a muscle can be computed quickly such that a response from the exoskeleton actuator can occur in real-time. An example of utilizing metabolic equations 1 through 5 in combination with the muscle-tendon interfaces is through the ultrasound interface. The ultrasound interface can measure muscle fiber length, velocity, and force using the methods outlined earlier. The Human Motor Intention Estimator 25 then uses this real-time estimate of muscle-tendon state, as well as muscle model coefficients, to estimate the total energy expenditure rate of one or multiple muscles. The metabolic energetics of the muscles are sent to the Augmentation Strategy Module 26. The Augmentation Strategy Module 26 will use the input to tune the gain or implement adaptive control to adjust according to the user's muscle energetic demands. For instance, if the ultrasound detects that the muscle is in a concentric, contracting state, the exoskeleton will apply positive torque to reduce the metabolic cost of the muscle in this inefficient regime.

In some example embodiments, the control diagram of FIG. 11 focuses on augmentation-based objectives for exoskeleton control using the power steering framework where muscle metabolic consumption serves as an input. First, the muscle-tendon state and current state of the exoskeleton 66 are estimated through the multiple Sensor Interfaces 67, the totality of the Muscle-Tendon Neural Interfaces and Intrinsic Sensors described earlier in FIGS. 2 through 10. Sensory information from the Sensor Interfaces is then input into the Muscle-Tendon State Estimator 68, a part of the Human Motor Intention Estimator from FIG. 1, to generate an estimation of the muscle metabolic power for those muscles of interest that span a joint or joints in parallel to the joint exoskeleton. The muscle metabolic power goes into an adaptive or constant functional relationship 69 which is performed by the Augmentation Strategies 70 computational module to produce an augmentation power or torque target. Augmentation Strategies include but are not limited to minimization of metabolic power of a muscle or muscles. Readings from the Sensor Interfaces are used by the Muscle-Tendon State Estimator to estimate biological muscle metabolic power in real time. With biological metabolic power as in input, the Augmentation Strategies then compute augmentation torque, or torques. The augmentation torque target is then input into the Torque Controller, which applies the torque to the human joint through a closed-loop wearable robotic control 71. On-board sensors of the wearable robot feedback the joint angle and output robotic torque back into the controller to close the loop on the augmentation torque command target 71. Without loss of generality, the augmentation torque determined by the Augmentation Strategy may be derived from a time-varying, joint equilibrium and impedance.

### Augmentation Objective-Based Control (Neuro-Optimal Control)

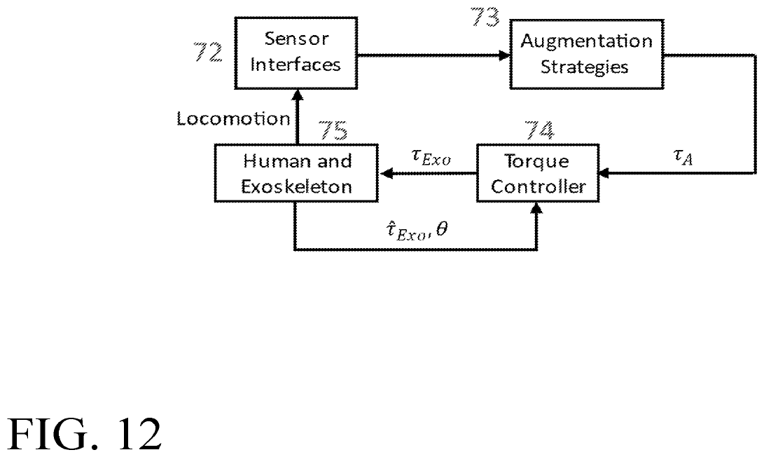

Another control architecture is one that uses augmentation objectives to compute the augmentation torque by adapting the augmentation torque profile in time to either minimize or maximize an objective function, rather than directly computing the augmentation torque from muscle-tendon state. This approach is described in FIGS. 12 and 13. For example, in FIG. 12 if the goal was to minimize the metabolic cost of walking, then measurements from the Sensor Interfaces 72 would inform the current state of the human and exoskeleton in which the Augmentation Strategies 73 can command a torque profile optimal to minimizing the metabolic cost given the current and past dynamics of the human. This augmentation torque profile updates when the output from the Sensor Interfaces 72 changes such that the current torque profile is no longer the most optimal in minimizing the cost function. This controller reflects a data-driven approach in which the sensory information is encoded as inputs into a function that determines the weight of each variable and its effect on the cost of the augmentation objective. The Augmentation Strategies 73 devises a torque profile, wherein the torque profile is changed by the weighted effects of the incoming sensory information. This augmentation torque profile is sent to the Torque Controller 74, which applies the torque to the human joint 75 through a closed-loop wearable robotic control. On-board sensors of the wearable robot feedback the joint angle and output robotic torque back into the controller to close the loop on the augmentation torque command target. Without loss of generality, the torque profile may be determined by modulating a joint equilibrium and impedance that minimizes the objective function.

Within FIG. 12 is the Augmentation Strategies 73, which consist of augmentation objectives to compute the augmented torque. Within these Augmentation Strategies, the signals from the Sensor Interfaces 76 are utilized to minimize the selected augmentation objective. In one embodiment shown in FIG. 13, the parameters from the Sensor Interfaces 76 are input into a Muscle Physiological Model 77 to estimate muscle-specific fatigue, metabolic rate, force transmission characteristics, these estimations are then used to compute either Global 78 or Muscle-Specific Objectives 80 to determine optimal assistive strategies. In some example embodiments, the Human Motor Intention Estimator comprises the Muscle Physiological Model 77. If sensory information includes multiple muscles, then the physiological indices increase to n number of muscles 79. If one muscle is the specific target to minimize its objective function such as muscle specific fatigue, then there will be a local minimum that provides augmentation to that muscle only. If n muscles are to be optimized, then all the muscles can be weighted and input to a Global Muscle Objective, representing the combination of objective functions of each muscle, and resulting in a global minimum across n muscles. In another embodiment, the parameters from the Sensor Interfaces 81 are used to estimate whole body metabolic rate through a feature extraction 82 to be used as an objective function for an optimal assistive strategy. In this data-driven example, sensory information details are extracted to estimate whole body metabolic rate, separate and distinct from measuring metabolic rate with, for example, a pulmonary gas exchange measurement unit. Another example is to use signals from acoustic or electric myography to get a frequency distribution and time series data that correlates to muscle fatigue and determines an assistive strategy that minimizes the muscle fatigue.

In summary, in FIG. 13 the parameters from the Sensor Interfaces 76 are input into a Muscle Physiological Model 77 to estimate muscle-specific fatigue, metabolic rate, force transmission characteristics, and these estimations are used to compute either global 78 or muscle-specific 80 objectives to determine optimal assistive strategies by the exoskeleton. In another embodiment, the parameters from the Sensor Interfaces 81 are used to estimate whole body metabolic rate 82, or individual muscle metabolism, to be used as an objective function for an optimal assistive strategy. Another embodiment is to use signals from acoustic myography to get a frequency distribution and time series data that correlates to muscle fatigue. Still further, in another embodiment, biomechanical objective functions, such as muscle CE power, can be used to generate an optimal torque profile. Overall, this control architecture can leverage the cost functions indicated in the below Objective Cost Functions section.

### Neuro-Reflexive Control

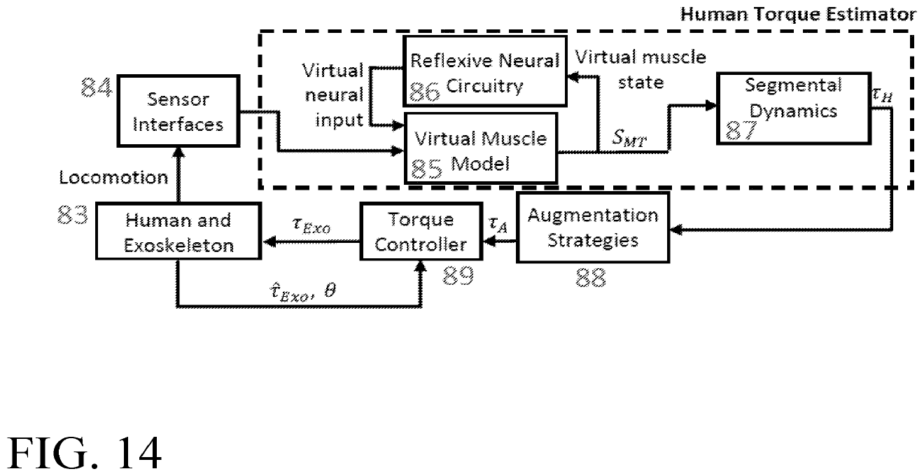

Another version of the Human Motor Intention Estimator is a reflexive architecture. In this architecture, the parameters from the muscle-tendon interfaces are input to a virtual neural circuitry model that computes virtual neural inputs to be used by a virtual muscle-tendon model. Parameters from the muscle-tendon interfaces are also used by the virtual muscle-tendon model to compute the intended muscle-tendon state parameters. This architecture is described in FIG. 14.

Specifically, parameters from the muscle-tendon interfaces serve as inputs to a reflexive neural circuitry model that computes a reflexive neural input that is later time-delayed. This signal, together with a volitional neural input from a volitional motor control decoder, serves as an input to a muscle activation dynamics model, which computes a virtual muscle activation. The activation together with parameters from the muscle-tendon interfaces are input into a segmental dynamics model to compute a torque, or joint equilibrium and impedance command target.

In the control diagram of FIG. 14, the setup is the same as the FIG. 11 control diagram, however, instead of human torque estimated directly from muscle-tendon neural signals and intrinsic sensors, the Human Torque Estimator estimates human biological torque using a reflexive architecture. The Virtual Muscle Model 85 receives muscle-tendon length and speed from the Sensor Interfaces 84 to compute a set of virtual muscle states. These virtual muscle states include outputs such as muscle-tendon force, activation, muscle length and velocity. These states can be input into the Reflexive Neural Circuitry 86 that in turn computes a virtual neural input that is fed back into the Virtual Muscle Model to establish a closed-loop reflex arc. The computed virtual muscle states, for example muscle-tendon force, can also be input directly into Segmental Dynamics 87 to compute the reflexive human torque output. This torque command is then sent to the Augmentation Strategies 88 computational module to estimate the augmentation torque. To apply the torque to the human wearer, the Torque Controller 89 then servos the wearable robot actuator 83 to the augmentation torque target using a closed-loop torque controller.

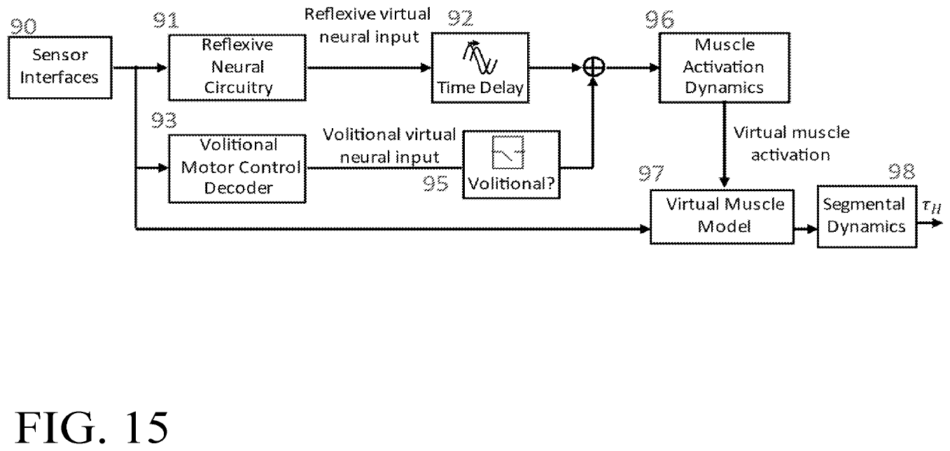

The embodiment of FIG. 15 expands upon FIG. 14 as this one allows for a volitional neural input to be used in combination with the reflexive neural input. In this architecture, the parameters from the Sensor Interfaces 90 are input into a Reflexive Neural Circuitry 91 module that computes reflexive virtual neural inputs, time delayed by the Time Delay module 92. The reflexive signals are combined with a volitional virtual neural input from a Volitional Motor Control Decoder 93, which computes sensory information into volitional input. Volitional input can be switched on or off depending on the activity 95. Both reflexive and volitional inputs are then sent to a Muscle Activation Dynamics module 96. This module computes a virtual muscle activation that together with parameters from the Sensor Interfaces are input into a Virtual Muscle Model 97. The Virtual Muscle Model computes virtual muscle states including muscle-tendon force. This signal is input into a Segmental Dynamics module 98 to compute the human biological torque. This control diagram represents another embodiment of the Human Motor Intention Estimator 25 described in FIG. 1. Without loss of generality, the Reflexive Neural Circuitry module may employ linear or non-linear, positive torque or displacement feedback to create the reflex behavior.

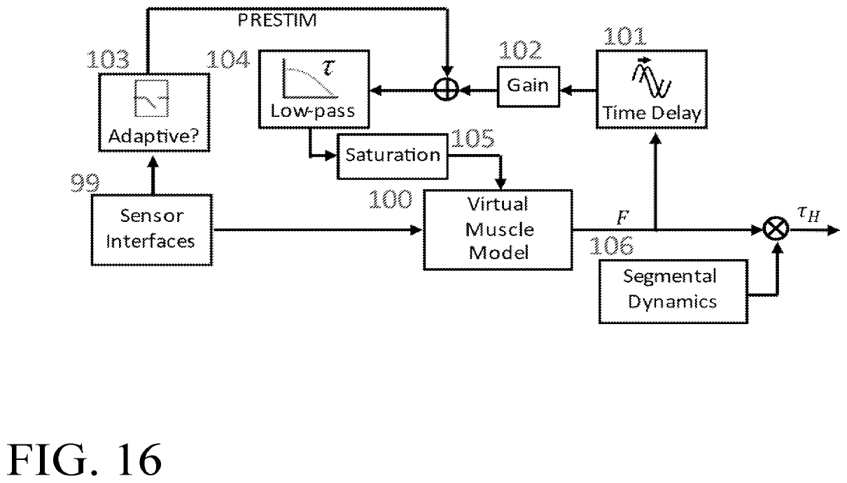

The FIG. 16 embodiment represents a particular reflexive control architecture using force feedback. In this architecture, parameters from the Sensor Interfaces 99, such as muscle length and speed, are input into a Virtual Muscle Model 100. This serves to trigger the force feedback model. This model computes muscle-tendon force that is time delayed via the Time Delay module 101, augmented by a Gain 102, low-pass filtered 104, normalized via the Saturation module 105, and input back to the Virtual Muscle Model 100. The possibility of a pre-stimulation value that can be constant or adaptive 103 based on parameters from the Sensor Interfaces can be added to the gain value. The muscle-tendon force estimate together with the Segmental Dynamics module 106 are used to compute the human torque. This architecture represents a particular form of reflexive control where force feedback is used to compute the reflexive input, which represents another embodiment of the Human Motor Intention Estimator 25 described in FIG. 1.

### Hybrid Controller for Exoskeleton Device Based on Uni-Directional Torque Actuator

In this section, we describe a low-level control architecture for an exoskeleton device utilizing a unidirectional torque actuator. In general, biological joints are bi-directional with agonist-antagonist muscle pairs for actuation. Thus, an exoskeleton device with a unidirectional torque actuator can cause discomfort or be limited in its efficacy. In such cases, one control approach is to do zero-impedance control for reverse direction actuation of the joint. For practical low-level controller designs, we describe herein a hybrid architecture in which torque control is applied in the forward direction while impedance control—with equilibrium being slacked—in the reverse direction. For example, a power-steering controller based on neural inputs can be applied in the forward direction while a slacked impedance control is applied in reverse. In one embodiment, EMG inputs from agonist and antagonist muscles spanning the ankle joint are used to calculate target ankle torque using an estimate of each muscle's length and speed, as well as each muscle-tendon's moment arm about the ankle joint. If the target is negative, the ankle operates in an impedance control mode with zero-impedance while adjusting the set-point to make the actuator operate in a slacked mode. In a special case where EMG is only measured from the agonist muscle, the controller first calculates the desired torque, and then evaluates the desired low-level device control mode based on torque thresholding. If the agonist torque is lower than the threshold, the device can behave in a zero-impedance mode with the equilibrium of the impedance controller equal to the current ankle position in order to enable joint slacking. When the agonist torque exceeds the threshold then the augmentation torque can be applied. This hybrid controller approach is a low-level control architecture that can be employed with the aforementioned high-level controllers.

### Objective Cost Functions

For the controllers mentioned earlier, user-specific torque profiles can be adapted to minimize a selected objective cost function. Below several examples are provided for cost functions.

### EMG

EMG signals indicate the target muscles' activation. In one embodiment, an exoskeleton control system varies its torque output so as to minimize muscle activation of one or more muscles. Here, EMG amplitude can be used as part of an objective cost function.

### Metabolic Rate and Muscle-Specific Fatigue

As noted previously, augmentation torque can be optimized to minimize a metabolic rate cost function, so as to reduce the tiredness of the user. Here the metabolic consumption of a single muscle or n muscles is computed in real time using the computational strategies outlined earlier. Further, a CE positive power cost function can be utilized as a proxy of metabolic rate. Still further, muscle fatigue can also serve as an objective cost function. Muscle fatigue information can be calculated by the combination of EMG signals, AMG signals, and ultrasound images. Muscle fatigue indicators can also be part of the exoskeletal cost function. For these metabolic rate and fatigue objective cost functions, the controller can prioritize exoskeletal augmentation when muscles are in their inefficient dynamic states (concentric contraction). This approach would allow for a re-organization of inefficient concentric and efficient eccentric muscle activities, improving the overall energy economy of movement.

### Muscle Efficiency

Augmentation torque can be optimized to maximize a muscle efficiency cost function. Here the muscle efficiency of a single muscle or n muscles is computed in real time using the computational strategies outlined earlier. As noted earlier, muscle efficiency can be computed directly as the ratio of mechanical power to metabolic power, or by measuring V/Vmax and using an a priori function that relates V/Vmax to muscle efficiency.

### Interaction Forces

The force between the exoskeleton and the user is a good indicator of the synergy of the two. Pressure readings from sensors can serve as the cost function to optimize the exoskeleton controller by minimizing the interaction forces. Compared to the metabolic energy and muscle fatigue, this optimization method can provide real-time feedback to fine tune the torque profile of the exoskeleton and ensure the exoskeleton does not resist the user's intended action.

### Human Center-of-Mass Stability

The user's center-of-mass trajectory can be calculated by inverse kinematics from the position sensors on the wearable robot. Center-of-Mass trajectory variation from biomechanical norms can also be part of the cost function to provide a smooth and stable experience for the user. Based on these parameters, the functions can minimize the maximum positive work done by each muscle while potentially increasing their negative work during full gait cycles. Another example of use is in the field of rehabilitation. For the use as a rehabilitation instrument, it is critical to provide augmentation based on the subject's physiological limits while maximizing the available neural input for successful rehabilitation outcomes. Having detailed neuromuscular information from the muscle-tendon interfaces, the exoskeleton assistance can be designed to support the specific muscle function based on the user's level of residual functionality.

FIG. 1 Overall system architecture

FIG. 1 shows different example embodiments of a robotic control system 200, in accordance with aspects of inventive concepts. In some example embodiments, the overall system architecture 200 comprises a wearable robot 22, one or more muscle-tendon neural interfaces 23, one or more intrinsic sensors 24, a Human Motor Intention Estimator 25, an Augmentation Strategy module 26, and a Torque Controller 27. A muscle-tendon neural interface may also be referred to as a muscle-tendon interface, a muscle-tendon sensor, or a sensor herein. A muscle-tendon interface can be an interface at one or more muscle, an interface at one or more tendon, or an interface at one or more muscle and one or more tendon.

In various embodiments, such as the one shown in FIG. 1, the wearable robot 22 is configured to be mounted at a user 21. In various embodiments, the one or more intrinsic sensors 24 are configured to collect muscle-tendon and joint physiological signals, including but not limited to muscle displacement, tendon displacement, muscle-tendon force, tendon wave speed, muscle stiffness, muscle electromyography, muscle velocity, tendon velocity, muscle-tendon unit (MTU) length, joint rotational position, rotational velocity, or rotational acceleration. The Human Motor Intention Estimator 25 then translates these signals to a real-time estimate of Muscle-Tendon State, including at least one of a muscle contractile element (CE) velocity, muscle-tendon unit (MTU) velocity, human biological muscle force, biological joint torque, biological CE mechanical power, muscle metabolic power, muscle efficiency, and muscle fatigue. To minimize muscle fatigue, reduce metabolic cost, increase muscle efficiency, or to amplify joint torque and power, the Augmentation Strategy 26 functionally relates the at least one of these Muscle-Tendon State signals to one or more augmentation torques applied about one or more robotic joints in real time and in an updating manner. For example, in one power-steering controller applied about a single degree of freedom (DOF) robotic joint, the augmentation torque applied about that DOF monotonically increases with increasing human biological joint torque about that DOF, using either a linear or non-linear functional relationship, to effectively amplify in real time muscle-tendon output torque. Additionally, sensory information from the Muscle-Tendon Neural Interfaces 23 and the Intrinsic Sensors 24 are inputted into the Augmentation Strategy computational module 26 directly to determine gait patterns or events, phase of gait, gait speed, and underlying terrain, and then the Augmentation Strategy 26 adapts the functional relationship between the at least one of the Muscle-Tendon State signals (biological torque, muscle CE power, etc.) and the one or more augmentation torques based upon these changing dynamics. Here, the augmentation torque(s) may be derived by a time-varying, joint equilibrium and impedance. The Torque Controller 27 then servos to these augmentation torques under computer control, applying the augmentation torques to human joints in 21 via the wearable-robot 22 in real time. To achieve this closed-loop, real-time control, the wearable robot 22 sends joint angle and torque information back to the Torque Controller 26 so that the augmentation torque modulation is adapted continuously. FIG. 1 shows different embodiments for a robotic control system 200. A specific embodiment may include one or more of the features and characteristics described. In some embodiments, for example the embodiment shown in FIG. 1, the control system 200 comprises a torque set point processor 201. In the example embodiment shown in FIG. 1, the torque set point processor 201 comprises the human motor intention estimator 25 and the augmentation strategy module 26. In alternative embodiments, the torque set point processor 201 may comprise one of the human motor intention estimator 25 or the augmentation strategy module 26. Alternatively, a common processor can be used by any one, two, or all three of the human motor intention estimator 25, the augmentation strategy module 26, or the torque controller 27.

### FIG. 2. Muscle-Tendon Interface: Ultrasound and Magnetic Bead Tracking

FIG. 2 shows different example embodiments for using ultrasound or magnetic bead tracking at a robotic control system 200, in accordance with aspects of inventive concepts. In some example embodiments, tracking markers are implanted within the muscle 10 and/or tendon 28. In some embodiments, two markers 11a, 11b are implanted into the tendon 28, and two markers 11c, 11d into the muscle 10, and the distance between each marker pair is computed in real time as an estimate of tendon and muscle displacements, respectively. The functional advantage of two markers compared to a single marker to estimate tendon or muscle displacements are that sensing errors caused by sensor-array movements on the skin can be mitigated.

In alternative embodiments, a different number of markers 11 may be implanted at the tendon 28. In alternative embodiments, a different number of markers 11 may be implanted at the muscle 10.

In some example embodiments, the one or more muscle-tendon interfaces 23 comprises one or more sensors 29. In some example embodiments, some of the one or more sensors 29 comprise sensor arrays. In some example embodiments, one or more of the one or more sensors 29 comprise ultrasound sensor arrays. In some example embodiments, one or more of the one or more sensors 29 comprise magnetic sensor arrays. For the tendon example, two markers are implanted and a skin-mounted sensor array 29 tracks the distance between the markers or the vibration of at least one marker. Using these data, muscle-tendon force can be estimated in one of two ways. In a first method, the distance between tendon marker pairs can be measured in real time to estimate tendon elongation, which when paired with an estimated or measured tendon stiffness value, directly correlates to muscle force 30, 31. In a second method, the Feature Extraction computational module 33 is used to estimate tendon vibration, which when coupled with the length of the tendon, correlates to muscle force 30, 33, 35. In the muscle example, two markers are implanted into the muscle 10 where the distance between the beads, the time rate of change of that distance, or velocity, is tracked 30, 32. Additionally, the vibration of at least one bead in the muscle can be estimated, which relates to muscle effort and activity 30, 33, 34. After information is collected relative to the markers within the muscle and tendon, they are sent to either Feature Extraction or directly correlate to length and velocity of the muscle. For example, if the markers' separation distance and velocity relative to each other are only being measured, then no feature extraction is required 30, 32. Feature Extraction is required in cases where the measurement from a marker or markers, such as vibrational response, needs to be processed to calculate the muscle activation in the case of muscle tissue 30, 33, 34, and muscle force in the case of tendon 30, 33, 35. In the case of muscle marker tracking, all the physiological parameters such as length, velocity, and activation are then input into a Muscle-Tendon Model 36, which outputs the muscle-tendon force 32, 34, 36. The muscle-tendon force from either of the three methods is then transmitted to Joint Segment Geometry 37 to calculate the output human torque at the target joint. In summary, there are three methods presented herein to estimate muscle-tendon force using the marker tracking technology, namely one method that relies on the muscle marker implants 32, 34, 36, 37, and two methods that rely on the tendon-marker implants 30, 31, 37; 30, 33, 35, 37. The system described above shows example embodiments of the Human Motor Intention Estimator 25 represented in FIG. 1.

FIG. 2 shows different embodiments for using ultrasound or magnetic bead tracking at an exoskeleton control system 200. A specific embodiment may include one or more of the features and characteristics described.

### FIG. 3. Muscle-Tendon Interface: Non-Invasive Ultrasound, EMG and Pressure.

FIG. 3 shows different example embodiments for using non-invasive techniques at a robotic control system 200, in accordance with aspects of inventive concepts. In the embodiments shown in FIG. 3 sensors 38, 39, 40 detect muscle-tendon state (length and velocity) and force without implanted markers. For example, sensors such as ultrasound 38 measure muscle length and velocity 44 through Image Processing 43 to analyze muscle fascicle state 38, 43, 44. This can be done through automatic edge and feature detection within the Image Processing system. The ultrasound sensors additionally enable Tissue Elastography 38, 41 which pulses the tissue and measures the response to estimate stiffness. This method targets the tendon to estimate tendon stiffness, which through a non-linear mapping, correlates to muscle-tendon force 38, 41, 42. The tracked muscle state from the ultrasound sensors can also be paired with a muscle activity measurement on the skin surface from either pressure/force 39, EMG 40 or ultrasound sensors 38. In some example embodiments, pressure/force sensors positioned between the skin surface adjacent the muscle, and an elastic sleeve that wraps around the limb circumferentially, can detect muscle contraction, volumetric and stiffness changes, relating to muscle activity 39, 45, 47. In some example embodiments, EMG sensors located on the skin surface can collect electric muscle activity adjacent the muscle 40, 45, 47. These signals pass through Feature Extraction 45 to process the raw signals from the sensors 39, 40 into clear muscle activity measurements. With multiple ultrasound sensors located around the muscle belly, an additional measurement from Image Processing includes muscle activity 47, which can be estimated through tracking changes in muscle volume 38, 43, 46, 47. The combination of muscle length, velocity and activation is fed into a Muscle-Tendon Model 48, which outputs a muscle-tendon force. The muscle-tendon force from either of the two methods is then transmitted to Joint Segment Geometry 49 to calculate the output biological torque at the target joint. In summary, there are two methods presented herein to estimate muscle-tendon force using non-invasive technology, namely one method that relies on Tissue Elastography and tendon stiffness 38, 41, 42, 49, and another which uses a combination of ultrasound imaging of muscle state and non-invasive muscle activity sensors 43, 44, 47, 48, 49. The system described above shows example embodiments of the Human Motor Intention Estimator 25 represented in FIG. 1.

FIG. 3 shows different embodiments for non-invasive techniques at an exoskeleton control system 200. A specific embodiment may include one or more of the features and characteristics described.

### FIG. 4. Muscle-Tendon Interface: Non-Invasive Ultrasound Doppler Velocimetry

FIG. 4A and FIG. 4B show an example embodiment of a first ultrasound generator 12, a second ultrasound generator 14, an ultrasound receiver 16, and an interference pattern, in accordance with aspects of inventive concepts. In some example embodiments, such as the one shown in FIG. 4A, two or more ultrasound generators with known and fixed frequency are positioned across either the muscle or tendon and focused to the tissue of interest. In the embodiment shown in FIG. 4A the first ultrasound generator 12 generates ultrasound at a frequency f. In the embodiment shown in FIG. 4A the second ultrasound generator 12 generates ultrasound at a frequency f+Δf. The focused ultrasound beams generate an interference pattern, such as the one shown in FIG. 4B and an ultrasound-based sensor receives the reflected wave from the tissue, with the incoming interference fringe frequency correlated to the tissue's velocity. The received signal can be processed through Fourier or wavelet transforms to determine the signal characteristics.

### FIG. 5. Muscle-Tendon Interface: Non-Invasive Ultrasound Doppler Velocimetry Signal Processing Pipeline

FIGS. 5A-5F show example embodiments of aspects of a non-invasive ultrasound Doppler velocimetry signal processing pipeline, in accordance with aspects of inventive concepts. Shown is the full pipeline (see FIG. 5A) from ultrasound receiver data collection to outputting the muscle-tendon velocity for the Human Motor Intention Estimator. The Hilbert transform (see FIG. 5B) is applied to generate an envelope of the received ultrasound signal. Afterwards, a bandpass filter is applied to the signal's power envelope (see FIG. 5C). To minimize the system delay and ensure frequency accuracy, a wavelet transform is applied on the band-passed envelope with the Morlet kernel (see FIG. 5D). The wavelet transformation plot is shown in FIG. 5D and the red box indicates the area selected to find the peak frequency. Finally, the frequency with the largest energy is found to be [symbol] (see FIG. 5E). By the equation [symbol] , the velocity is calculated. The main frequency can also be found in the FFT method (see FIG. 5F), but this is limited by the window size which determines the minimum discernible frequency.

### FIG. 6. Muscle-Tendon Interface: Non-Invasive Ultrasound Doppler Velocimetry Designs

FIG. 6A shows an example embodiment of a first ultrasound generator 12, a second ultrasound generator 14, an ultrasound receiver 16, and a layer of ultrasound transparent material 15, in accordance with aspects of inventive concepts. In the embodiment shown in FIG. 4A the first ultrasound generator 12 generates ultrasound at a frequency f. In the embodiment shown in FIG. 6A the second ultrasound generator 12 generates ultrasound at a frequency f+Δf. In some example embodiments, such as the one shown in FIG. 6A, two ultrasound generators are placed on an ultrasound transparent material with a fixed angle. This material configuration allows the generators 12, 14 to be placed at an angle, so the beams intersect.

FIG. 6B shows an example embodiment of a first ultrasound generator 12, a second ultrasound generator 14, an ultrasound receiver 16, a layer of skin and fat 2, a layer of muscle 10 and tendon 28, and a layer of bone 4, in accordance with aspects of inventive concepts. In this embodiment, the ultrasound beams interfere in muscle/tendon 10, 28, and the backscattered signals are collected by the receiver 16.

### FIG. 7. Muscle-Tendon Interface: Non-Invasive Ultrasound Doppler Velocimetry Generator Designs

FIG. 7A shows an example embodiment of an ultrasound generator 12, 14 with acoustic holographic layer 17 on top of it, in accordance with aspects of inventive concepts. In some example embodiments, more than one acoustic holographic layer 17 may be mounted on an ultrasound generator 12, 14. The holographic layers 17 on each ultrasound generator 12, 14 allow for steering and focusing the ultrasound beam to the surface of the muscle.

FIG. 7B shows an example embodiment of an ultrasound generator 12, 14 coupled to an acoustic holographic layer 17 and a gel pad 15, in accordance with aspects of inventive concepts. All of the sensors (generators and/or detectors) can be placed on the skin flatly with sticky gel pads 15.

### FIG. 8. Muscle-Tendon Interface: Non-Invasive Ultrasound Doppler Velocimetry Interference Diagrams

FIG. 8A shows an example embodiment of interference fringes movement along with muscle/tendon movement, in accordance with aspects of inventive concepts.

FIG. 8B shows an example embodiment of interference fringes, an ultrasound receiver 16, and an acoustic lens system 18, in accordance with aspects of inventive concepts. In some example embodiments, the ultrasound receiver 16 can be, but is not limited to, a single piezoelectric material or a PZT or capacitor array. To minimize the interference between ultrasound signals from backscatters, an acoustic lens 18 can be placed between the ultrasound receiver and the ultrasound gel pad 15 on the skin. The acoustic lens 18 ensures that the center plane of the interference area focuses on the receiver, as shown in FIG. 8B.

### FIG. 9. Muscle-Tendon Interface: Non-Invasive Acoustic, Strain and Impulse Generator Sensing and Actuation

FIG. 9 shows example embodiments of non-invasive acoustic, strain, and impulse generator sensing and actuation systems, devices, and methods, in accordance with aspects of inventive concepts. As described herein, sensing and actuation methods are used in unique combinations to estimate human joint torque. Acoustic and strain sensors such as microphone transducers or strain fibers can capture the muscle and tendon's response to external or internal disturbances 50, and when paired with an impulse generator 51 can collect tissue stiffness. The impulse generator 51 is a tissue actuator that applies a repeated force palpation to the tissue at a known frequency using either a vibratory motor, an ultrasound unit, or any other actuator detailed in the specification. This repeated palpation actuation creates a shear-wave propagating in the tissue that can be measured with a microphone transducer or strain fibers 50 and correlated to muscle force through a Shear Wave Model 52. This model includes the basic physiological parameters of the tissue, such as modulus, density, and surface area to provide an accurate estimation of muscle-tendon force from a propagated shear wave.

In some embodiments, a vibrational motor actuates the tendon, creating shear waves which are then recorded by acoustic transducers and input into the Shear Wave Model 52 to generate muscle-tendon force 50, 51, 52, 53. In another embodiment, the ultrasound unit creates an internal focused pressure wave within the tissue to generate an impulse, and with subsequent imaging, records the wave speed through the tissue 50, 51, 52, 53. In another embodiment, the impulse generator 51 can estimate the stiffness of the muscle through frequent actuator palpations with real-time measurements of palpation force and tissue displacements caused by the actuator as the muscle-tendon is dynamically activated by the nervous system. This estimated muscle stiffness has a non-linear mapping to the force the muscle generates 51,54. These muscle-tendon forces are input to the Joint Segment Geometry 55 to calculate the output human torque at the target joint. In summary, there are two methods presented herein to estimate muscle-tendon force using non-invasive technology, namely one method that relies on constant palpation to estimate muscle stiffness 51, 54, 55, and another which uses shear wave propagation 50, 52, 53, 55. The system described shows some example embodiments of the Human Motor Intention Estimator 25 represented in FIG. 1.

FIG. 9 shows different embodiments of non-invasive acoustic, strain, and impulse generator sensing and actuation systems, devices, and methods. A specific embodiment may include one or more of the features and characteristics described.

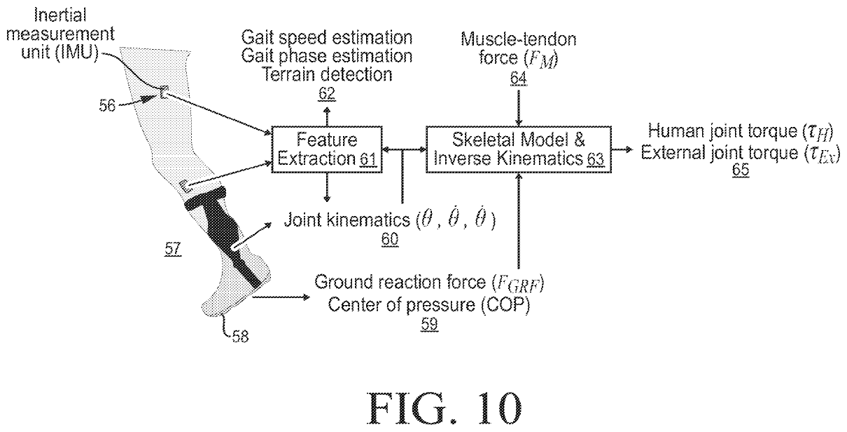

### FIG. 10. Muscle-Tendon Interface: Non-Invasive Kinetic and Kinematic Sensing

FIG. 10 shows example embodiments of non-invasive kinetic and kinematic sensing systems, devices, and methods, in accordance with aspects of inventive concepts. Inertial measurement unit (IMU) and kinetic-based sensors are used to estimate joint torque using an inverse-dynamics calculation. IMUs attached to the body at known anatomical locations 56 can be used to estimate joint kinematics in real time 57, 60. Force/pressure sensors on the sole of the foot 58 measure ground reaction forces and center-of-pressure at the foot-ground interface 59. The IMU-based measurements, combined with the force/pressure measurements, are processed through Feature Extraction 61 and machine learning techniques to output joint kinematics, gait events, gait phase, gait speed, and underlying terrain characteristics 56, 57, 60, 61, 62. The IMU joint kinematics, ground reaction forces, and center of pressure are fed into a Skeletal Model 63, and inverse dynamics are performed to estimate either a biological human torque or a net external joint torque 60, 63, 65). If the force/pressure sensors are positioned between the human limb and the exoskeleton, the biological human torque can be estimated 58, 59, 63, 65. In distinction, if the force/pressure sensors are positioned between the exoskeleton and the interaction surface, the total net external joint torque can be estimated from the inverse dynamics calculation 58, 59, 63, 65. The muscle-tendon force from other sensing modalities can be input into the Skeletal Model to produce a human joint torque 64, 63, 65. By subtracting the exoskeletal joint torque from the net joint torque, the human torque can be estimated, which acts as the Human Motor Intention Estimator 25 from FIG. 1. The estimation of gait events, gait phase, gait speed, and underlying terrain characteristics 62 are then used by the Augmentation Strategy module 26 shown in FIG. 1 to update the functional relationship between human torque and augmentation torque.

FIG. 10 shows different embodiments of non-invasive kinetic and kinematic sensing systems, devices, and methods. A specific embodiment may include one or more of the features and characteristics described.

### FIG. 11. Control Architecture Embodiment: Power Steering Control

FIG. 11 shows an example embodiment of a control architecture for power steering control, in accordance with aspects of inventive concepts.

### FIG. 12. Control Architecture Embodiment: Augmentation Objective-Based Control

FIG. 12 shows an example embodiment of a control architecture for augmentation objective-based control, in accordance with aspects of inventive concepts.

### FIG. 13. Objective Functions for Augmentation Strategies

FIG. 13 shows example objective functions for augmentation strategies, in accordance with aspects of inventive concepts.

### FIG. 14. Control Architecture Embodiment: Reflexive Control

FIG. 14 shows an example embodiment of a control architecture for reflexive control, in accordance with aspects of inventive concepts.

### FIG. 15. Control Architecture Embodiment: Reflexive and Volitional Control

FIG. 15 shows an example embodiment of a control architecture for reflexive and volitional control, in accordance with aspects of inventive concepts.

### FIG. 16. Control Architecture Embodiment: Reflexive Control Using Force Feedback

FIG. 16 shows an example embodiment of a control architecture for reflexive control using force feedback, in accordance with aspects of inventive concepts.

### REFERENCES

1. US20210100460A1, Conformable Garment for Physiological Sensing
2. U.S. Pat. No. 9,975,249B2, Neuromuscular model-based sensing and control paradigm for a robotic leg
3. US20200276698A1. Controls Optimization for Wearable Systems. https://patents.google.com/US20200276698A1/en
4. US 2017/0202724 A1. ASSISTIVE FLEXIBLE SUITS, FLEXIBLE SUIT SYSTEMS, AND METHODS FOR MAKING AND CONTROL THEREOF TO ASSIST HUMAN MOBILITY (https://patentimages.storage.googleapis.com/51/2d/a0/5ac7fba3292c6a/US20170202724
5. U.S. Pat. No. 10,843,332B2. Soft exosuit for assistance with human motion (https://patentimages.storage.googleapis.com/8c/30/82/8ba63ddf1c8a56/US10843332.pdf)
6. US 2021/0039248 A1. Soft exosuit for assistance with human motion (https://patentimages.storage.googleapis.com/e2/ee/2f/3d001d2c4c6a56/US20210039248 A1.pdf)
7. U.S. Pat. No. 9,351,900B2. Soft exosuit for assistance with human motion (https://patents.google.com/patent/US9351900B2/en)
8. U.S. Pat. No. 10,555,865. Torque control methods for an exoskeleton device. https://patentimages.storage.googleapis.com/6a/23/b0/b6259553f87ba0/US10555865.pdf
9. US 2019/0200900 A. APPARATUS FOR INTRAOPERATIVE LIGAMENT LOAD MEASUREMENTS (https://patentimages.storage.googleapis.com/e2/13/f2/a60faa0e586094/US20190200900 A1.pdf)
10. Nuckols, Richard W., et al. “Individualization of exosuit assistance based on measured muscle dynamics during versatile walking.” Science robotics 6.60 (2021):
11. Baud, Romain, et al. “Review of control strategies for lower-limb exoskeletons to assist gait.” Journal of NeuroEngineering and Rehabilitation 18.1 (2021): 1-34.
12. https://journals.plos.org/plosone/article?id=10.1371/journal.pone.0246611
13. https://ieeexplore.ieee.org/stamp/stamp.jsp?arnumber=9521161
14. Brian R Umberger. Stance and swing phase costs in human walking. Journal of the Royal Society, Interface/the Royal Society, 7(50):1329 {40, September 2010.
15. Brian R Umberger, Karin G M Gerritsen, and Philip E Martin. A model of human muscle energy expenditure. Computer methods in biomechanics and biomedical engineering, 6(2):99 {111, April 2003.
16. A more precise, repeatable and diagnostic alternative to surface electromyography—an appraisal of the clinical utility of acoustic myography. https://onlinelibrary.wiley.com/doi/full/10.1111/cpf.12417
17. Muscle-related differences in mechanomyography-force relationships are model-dependent. https://onlinelibrary.wiley.com/doi/10.1002/mus.23896
18. S. H. Yeon, H. Song and H. M. Herr, “Spatiotemporally Synchronized Surface EMG and Ultrasonography Measurement Using a Flexible and Low-Profile EMG Electrode,” 2021 43rd Annual International Conference of the IEEE Engineering in Medicine & Biology Society (EMBC), 2021, pp. 6242-6246, doi: 10.1109/EMBC46164.2021.9629789.

The teachings of all patents, published applications and references cited herein are 1835 incorporated by reference in their entirety.

The various features described above can be used separately and in combination.

In some embodiments, the term “real time” herein refers to times within 100 ms.

In some embodiments, at least a portion of one or more of the modules discussed herein (for example, the human motor intention estimator 25, the augmentation strategy module 26, the torque controller 27) may be implemented with software.

In some embodiments, at least a portion of one or more of the modules discussed herein (for example, the human motor intention estimator 25, the augmentation strategy module 26, the torque controller 27) may be implemented with one or more processors.

In some embodiments, at least a portion of one or more of the modules discussed herein (for example, the human motor intention estimator 25, the augmentation strategy module 26, the torque controller 27) may be stored on one or more storage mediums.

While example embodiments have been particularly shown and described, it will be understood by those skilled in the art that various changes in form and details may be made therein without departing from the scope of the embodiments encompassed by the appended claims.

What is claimed is:

1.-31. (canceled)

32. A wearable robotic control system, comprising:
at least one actuated joint;
at least one muscle-tendon interface configured to measure at least one physiological signal;
a processor configured to:
receive the at least one physiological signal from the at least one muscle-tendon interface and estimate a muscle-tendon state, the muscle-tendon state corresponding to a human motor intention, the muscle-tendon state being at least one of a muscle-tendon length, a muscle-tendon speed, a muscle-tendon impedance, or a muscle-tendon force; and
compute in a continuous and real time manner at least one augmentation joint command based on the muscle-tendon state; and
a controller configured to apply the at least one augmentation joint command to the at least one actuated joint.

33. The wearable robotic control system of claim 32, wherein the at least one augmentation joint command comprises at least one of joint torque, joint impedance, or joint position.

34. The wearable robotic control system of claim 32, wherein the controller is configured to apply the at least one augmentation joint command to the at least one actuated joint of the wearable robot within 100 ms of the at least one muscle-tendon interface measuring the at least one physiological signal.

35. The wearable robotic control system of claim 32, wherein the processor comprises:
a human motor intention estimator configured to receive the at least one physiological signal from the at least one muscle-tendon interface and estimate the muscle-tendon state; and
an augmentation strategy module configured to receive the estimate of the muscle-tendon state and compute the at least one augmentation joint command.

36. The wearable robotic control system of claim 35, wherein the at least one muscle-tendon interface comprises at least one ultrasound probe and the human motor intention estimator comprises a tissue elastography module, the tissue elastography module being configured to:
pulse a tissue using an actuator on the outside of skin, or pulse the tissue using an acoustic radiation force impulse from an ultrasound system, and
receive information from the at least one ultrasound probe to measure a tissue elasticity response.

37. The wearable robotic control system of claim 36, wherein the human motor intention estimator is configured to use a shear wave on a muscle or a tendon to estimate muscle stiffness or tendon stiffness.

38. The wearable robotic control system of claim 37, wherein the human motor intention estimator is configured to use the muscle stiffness or the tendon stiffness to estimate a muscle-tendon force.

39. The wearable robotic control system of claim 35, wherein the at least one muscle-tendon interface comprises at least one ultrasound probe and the human motor intention estimator is configured to use a muscle vibration measurement to estimate a muscle activation.

40. The wearable robotic control system of claim 35, wherein the human motor intention estimator comprises an image processing module configured to analyze ultrasound images using at least one of optical flow algorithms, edge detection, filtering, or neural networks to estimate the muscle-tendon state.

41. The wearable robotic control system of claim 39, wherein the muscle-tendon force is estimated using the muscle activation, a muscle length, and a muscle velocity.

42. The wearable robotic control system of claim 32, wherein the at least one muscle-tendon interface comprises:
at least two ultrasound generators configured to emit ultrasound waves toward a muscle-tendon target at different specific frequencies;
an ultrasound receiver configured to receive ultrasound waves reflected from the muscle-tendon target, wherein an intensity of the ultrasound waves changes periodically at a frequency correlated with muscle or tendon moving velocity.

43. The wearable robotic control system of claim 42, wherein an acoustic lens is coupled to the ultrasound receiver.

44. The wearable robotic control system of claim 35, wherein the augmentation strategy module is configured to compute the at least one augmentation joint command in real time.

45. The wearable robotic control system of claim 35, wherein the augmentation strategy module is configured to compute the at least one augmentation joint command in real time, the augmentation joint command being configured to monotonically increase or decrease with increases or decreases in its corresponding biological muscle torque, muscle power, or muscle metabolic power.

46. The wearable robotic control system of claim 35, wherein the augmentation strategy module adjusts the at least one augmentation joint command to optimize the muscle-tendon state to reduce metabolic cost or maximize muscle efficiency for at least one muscle.

47. The wearable robotic control system of claim 32, further comprising:
an ultrasound probe configured to measure at least one physiological signal; and
an image processing module configured to estimate muscle length or muscle velocity from an ultrasound image using at least one of optical flow algorithms, edge detection, filtering, affine transformation, or neural networks to estimate the muscle-tendon state.

48. The wearable robotic control system of claim 47, wherein the at least one muscle-tendon interface comprises at least two markers within a muscle or tendon and at least one ultrasound sensor and the human motor intention estimator comprises a marker tracking module configured to receive information from the at least one ultrasound sensor and measure a distance and a velocity between the at least two markers to estimate a muscle-tendon length and a muscle-tendon velocity.

49. The wearable robotic control system of claim 32, wherein the at least one muscle-tendon interface comprises:
at least one marker implanted within a muscle-tendon;
at least one ultrasound sensor; and
a marker tracking module configured to receive information from the at least one ultrasound sensor and estimate a muscle or tendon wave speed or vibration frequency as an indicator of muscle-tendon force.

50. The wearable robotic control system of claim 32, wherein the at least one muscle-tendon interface comprises:
an impulse generator configured to vibrate a tendon or muscle, thereby generating a shear wave; and
at least one acoustic sensor configured to be positioned on a skin surface, the at least one acoustic sensor being configured to measure a speed of the shear wave, the speed of the shear wave being used to estimate a muscle activation, the muscle activation being the physiological signal.

51. A method to control at least one actuated joint of a wearable robot, comprising:
measuring at least one physiological signal using at least one muscle-tendon interface;
estimating a muscle-tendon state, the muscle-tendon state corresponding to a human motor intention, the muscle-tendon state being at least one of a muscle-tendon length, a muscle-tendon speed, a muscle-tendon impedance, or a muscle-tendon force;
computing at least one augmentation joint command based on the muscle-tendon state; and
applying the at least one augmentation joint command to the at least one actuated joint of the wearable robot.
